(19) United States
(12) Patent Application Publication
(10) Pub. No.: US 2006/0004036 A1
(43) Pub. Date: Jan. 5, 2006

(54) Sigma-2 receptor agonists and their use in the treatment of HIV infection

(75) Inventors: Wayne D. Bowen, Cumberland, RI (US); Keith W. Crawford, Washinton, DC (US); James E. Hildreth, Brentwood, TN (US)

Correspondence Address: BERENATO, WHITE & STAVISH, LLC, 6550 ROCK SPRING DR., SUITE 240, BETHESDA, MD, 20817, US

(73) Assignee: The United States of America, per Secy. DHHS, NIH, Rockville, MD (US)

(21) Appl. No.: 11/181,797

(22) Filed: Jul. 15, 2005

Related U.S. Application Data
(63) Continuation-in-part of application No. PCT/US04/00739, filed on Jan. 14, 2004.
(60) Provisional application No. 60/440,367, filed on Jan. 16, 2003.

(51) Int. Cl. A61K 31/485

(52) U.S. Cl. 514/282

(57) ABSTRACT

The present invention relates to sigma-2 receptor agonists and their use in the treatment of immunodeficiency virus infections, especially human immunodeficiency virus (HIV) infections. The invention particularly relates to sigma-2 agonists, especially CB-184 and its analogues, that decrease cellular production of sphingomyelin when provided to recipient cells, and inhibit HIV replication.

### CROSS-REFERENCE TO RELATED APPLICATIONS

This application is a continuation-in-part of PCT Application PCT/U.S. 2004/000739, filed Jan. 14, 2004 (pending); this application claims priority to PCT Application PCT/US2004/000739, filed Jan. 14, 2004, and U.S. Patent Application Ser. No. 60/440,367, filed on Jan. 16, 2003, both of which applications are hereby incorporated by reference in their entirety.

### STATEMENT OF GOVERNMENTAL INTEREST

Aspects of the invention were funded by the United States National Institutes of Health. The United States Government has certain rights to this invention.

### FIELD OF THE INVENTION

This invention relates to sigma-2 receptor agonists and their use in the treatment of immunodeficiency virus infections, especially human immunodeficiency virus (HIV) infections.

### BACKGROUND OF THE INVENTION

The human immunodeficiency virus (HIV) is a pathogenic retrovirus (Varmus, H. (1988) “RETROVIRUSES,” Science 240:1427-1439; Cowley S. (2001) “THE BIOLOGY OF HIV INFECTION” Lepr Rev. 72(2):212-20). It is the causative agent of acquired immune deficiency syndrome (AIDS) and related disorders (Gallo, R. C. et al. (1983) “Isolation of human T-cell leukemia virus in acquired immune deficiency syndrome (AIDS),” Science 220(4599):865-7; Barre-Sinoussi, F. et al. “ISOLATION OF A T-LYMPHOTROPIC RETROVIRUS FROM A PATIENT AT RISK FOR ACQUIRED IMMUNE DEFICIENCY SYNDROME (AIDS), (1983) Science 220:868-870; Gallo, R. et al. (1984) “FREQUENT DETECTION AND ISOLATION OF CYTOPATHIC RETROVIRUSES (HTLV-III) FROM PATIENTS WITH AIDS AND AT RISK FOR AIDS,” Science 224:500-503; Teich, N. et al. (1984) “RNA TUMOR VIRUSES,” Weiss, R. et al. (eds.) Cold Spring Harbor Press (NY) pp. 949-956). HIV acts to compromise the immune system of infected individuals by targeting and infecting the CD-4⁺ T lymphocytes that would otherwise be the major proponents of the recipient's cellular immune system response (Dalgleish, A. et al. (1984) “THE CD4 (T4) ANTIGEN IS AN ESSENTIAL COMPONENT OF THE RECEPTOR FOR THE AIDS RETROVIRUS,” Nature 312: 767-768, Maddon et al. (1986) “THE T4 GENE ENCODES THE AIDS VIRUS RECEPTOR AND IS EXPRESSED IN THE IMMUNE SYSTEM AND THE BRAIN,” Cell 47:333-348; McDougal, J. S. et al. (1986) “BINDING OF HTLV-III/LAV TO T4+ T CELLS BY A COMPLEX OF THE 110 K VIRAL PROTEIN AND THE T4 MOLECULE,” Science 231:382-385). HIV infection is pandemic and HIV-associated diseases represent a major world health problem.

Attempts to treat HIV infection have focused on the development of drugs that disrupt the viral infection and replication cycle (see, Mitsuya, H. et al. (1991) “TARGETED THERAPY OF HUMAN IMMUNODEFICIENCY VIRUS-RELATED DISEASE,” FASEB J. 5:2369-2381). Such intervention could potentially inhibit the binding of HIV to cell membranes, the reverse transcription of the HIV RNA genome into DNA, the exit of the virus from the host cell and infection of new cellular targets, or inhibition of viral enzymes (see, U.S. Pat. No. 6,475,491). Thus, for example, soluble CD4 has been developed in an effort to competitively block the binding of HIV to lymphocytes (Smith, D. H. et al. (1987) “BLOCKING OF HIV-1 INFECTIVITY BY A SOLUBLE, SECRETED FORM OF THE CD4 ANTIGEN,” Science 238:1704-1707; Schooley, R. et al. (1990) “RECOMBINANT SOLUBLE CD4 THERAPY IN PATIENTS WITH THE ACQUIRED IMMUNODEFICIENCY SYNDROME (AIDS) AND AIDS-RELATED COMPLEX. A PHASE I-II ESCALATING DOSAGE TRIAL,” Ann. Int. Med. 112:247-253; Kahn, J. O. et al. (1990) “THE SAFETY AND PHARMACOKINETICS OF RECOMBINANT SOLUBLE CD4 (RCD4) IN SUBJECTS WITH THE ACQUIRED IMMUNODEFICIENCY SYNDROME (AIDS) AND AIDS-RELATED COMPLEX. A PHASE I STUDY,” Ann. Int. Med. 112:254-261; Yarchoan, R. et al. (1989) Proc. V^{th} Int. Conf. on AIDS, p564, MCP 137).

Likewise, drugs such as 2′,3′-dideoxynucleoside analogs (e.g., azidothymidine (AZT), dideoxyinosine, dideoxycytidine, and d4thymidine have been developed to target the virus' reverse-transcriptase (Yarchoan, R. et al. (1989) “CLINICAL PHARMACOLOGY OF 3′-AZIDO-2′,3′-DIDEOXYTHYMIDINE (ZIDOVUDINE) AND RELATED DIDEOXYNUCLEOSIDES,” N Engl J. Med. 321(11):726-38). Recently, inhibitors of the HIV protease have been identified and used to treat HIV infection (see, U.S. Pat. No. 6,472,404; Todd, S. et al. (2000) “HIV PROTEASE AS A TARGET FOR RETROVIRUS VECTOR-MEDIATED GENE THERAPY,” Biochim Biophys Acta. 1477(1-2):168-88).

Unfortunately, although considerable effort has been expended to design effective therapeutics, no curative anti-retroviral drugs against AIDS currently exist. All available therapies are marred by substantial adverse side effects, and by the capacity of HIV to rapidly mutate into forms that are refractive to treatment (Miller, V. et al. (2001) MUTATIONAL PATTERNS IN THE HIV GENOME AND CROSS-RESISTANCE FOLLOWING NUCLEOSIDE AND NUCLEOTIDE ANALOGUE DRUG EXPOSURE,” Antivir Ther. 6 Suppl 3:25-44; Lerma, J. G. et al. (2001) “RESISTANCE OF HUMAN IMMUNODEFICIENCY VIRUS TYPE I TO REVERSE TRANSCRIPTASE AND PROTEASE INHIBITORS: GENOTYPIC AND PHENOTYPIC TESTING,” J Clin Virol. 21(3): 197-212; O'Brien, W. A. (2000) “RESISTANCE AGAINST REVERSE TRANSCRIPTASE INHIBITORS,” Clin Infect Dis. 30 Suppl 2:S185-92; Wain-Hobson, S. (1996) “RUNNING THE GAMUT OF RETROVIRAL VARIATION,” Trends Microbiol. 4(4):135-41; Lange J. (1995) “COMBINATION ANTIRETROVIRALTHERAPY. BACK TO THE FUTURE,” Drugs. 49 Suppl 1:32-40). Thus, a continuing need exists for safe and effective anti-HIV therapeutics. The present invention is directed to this and other goals.

Sigma receptors are unique drug binding sites with pharmacological profiles and potential functions that are unrelated to other known receptors (Walker, J. M. et al. (1990) “SIGMA RECEPTORS: BIOLOGY AND FUNCTION,” Pharmacol. Rev. 42:355-402; Su, T.-P. (1993) “DELINEATING BIOCHEMICAL AND FUNCTIONAL PROPERTIES OF SIGMA RECEPTORS: EMERGING CONCEPTS,” Crit. Rev. Neurobiol. 7:187-203; Bowen, W. D. (2000) “SIGMA RECEPTORS: RECENT ADVANCES AND NEW CLINICAL POTENTIALS,” Pharm. Acta Helv. 74(2-3):211-8). Two pharmacologically defined subclasses of sigma-receptor (sigma-1 and sigma-2) are now widely accepted (Hellewell, S. B. et al. (1990) “A SIGMA-LIKE BINDING SITE IN RAT PHEOCHROMOCYTOMA (PC12) CELLS: DECREASED AFFINITY FOR (+)-BENZOMORPHANS AND LOWER MOLECULAR WEIGHT SUGGEST A DIFFERENT SIGMA RECEPTOR FORM FROM THAT IN GUINEA PIG BRAIN,” Brain Res. 527: 244-253; Quirion, R. et al. (1992) “A PROPOSAL FOR THE CLASSIFICATION OF SIGMA BINDING SITES,” Trends Pharmacol. Sci. 13: 85-86). Sigma receptors are expressed in many cell types and tissues, including those of the nervous, immune, and endocrine systems (Wolfe, S. A. et al. (1989) “SIGMA RECEPTORS IN ENDOCRINE ORGANS—IDENTIFICATION, CHARACTERIZATION, AND AUTORADIOGRAPHIC LOCALIZATION IN RAT PITUITARY, ADRENAL, TESTIS, AND OVARY,” Endocrinol. 124:1160-1172; Wolfe, S. A. et al. (1992) “SIGMA RECEPTORS IN THE BRAIN-ENDOCRINE-IMMUNE AXIS,” In: J. M. Kamenka and E. F. Domino (eds.), MULTIPLE SIGMA AND PCP RECEPTOR LIGANDS: MECHANISMS FOR NEUROMODULATION AND NEUROPROTECTION?, pp. 115-126, Ann Arbor: NPP Books; Hellewell, S. B. et al. (1994) “RAT LIVER AND KIDNEY CONTAIN HIGH DENSITIES OF SIGMA-1 AND SIGMA-2 RECEPTORS: CHARACTERIZATION BY LIGAND BINDING AND PHOTOAFFINITY LABELING,” Eur. J. Pharmacol.—Mol. Pharmacol. Sect. 268:9-18) Furthermore, sigma receptors are highly expressed in tumor cell lines derived from these tissues (Vilner, B. J. et al. (1995) “SIGMA-1 AND SIGMA-2 RECEPTORS ARE EXPRESSED IN A WIDE VARIETY OF HUMAN AND RODENT TUMOR CELL LINES,” Cancer Res. 55:408-413) and are upregulated in rapidly proliferating cells (Wheeler, K. T. et al. (2000) “SIGMA-2 RECEPTORS AS A BIOMARKER OF PROLIFERATION IN SOLID TUMOURS.” Br. J. Cancer 82(6): 1223-32).

Sphingolipids include classes of lipids that constitute major structural components of specialized domains in cell membranes (Brown, D. A. et al. (2000) “STRUCTURE AND FUNCTION OF SPHINGOLIPID- AND CHOLESTEROL-RICH MEMBRANE RAFTS,” J. Biol. Chem. 275:17221-17224) Additionally, certain sphingolipids and sphingolipid-derived compounds have been found to affect cell growth and proliferation, as well as cell death (Kolesnick, R. N. et al. (1998) “REGULATION OF CERAMIDE PRODUCTION AND APOPTOSIS,”Annu. Rev. Physiol. 60:643-665). Sigma-2 receptor activation has an effect on cellular proliferation and survival which may be mediated by sphingolipids.

Compounds that activate the sigma-2 receptor (i.e., “sigma-2 agonists”) have been identified (PCT Publication No. WO 01/85153; PCT Publication No. WO 01/80905; PCT Publication No. WO 97/34892; PCT Publication No. WO 97/30038; PCT Publication No. WO 96/05185; European Patent Publication No. EP 0881220A1; U.S. Pat. No. 6,015,543; U.S. Pat. No. 5,993,777; U.S. Pat. No. 5,919,934; U.S. Pat. No. 5,969,138; U.S. Pat. No. 5,911,970.

Some sigma-2 receptor agonists have been found to increase cellular ceramide production. Such compounds are generally recognized as cytotoxic, and are used to potentiate the use of antineoplastic agents in the treatment of cancer cells (see, PCT Publication No. WO 01/85153; Crawford, K. W. et al. (2002) “SIGMA-2 RECEPTOR AGONISTS ACTIVATE A NOVEL APOPTOTIC PATHWAY AND POTENTIATE ANTINEOPLASTIC DRUGS IN BREAST TUMOR CELL LINES,” Cancer Res 62(1):313-22). The use of sigma-2 agonists to regulate sphingolipid levels is disclosed in Crawford, K. W. et al. (2002) (“SIGMA-2 RECEPTORS REGULATE CHANGES IN SPHINGOLIPID LEVELS IN BREAST TUMOR CELLS,” Eur. J. Pharmacol. 443:207-209), Crawford, K. W. et al. (2001) (“SIGMA-2 RECEPTORS MAY ACTIVATE SPHINGOLIPID-CERAMIDE N-DEACYLASE (SCDASE) AS A MECHANISM TO REGULATE CELL GROWTH,” Soc. Neurosci. Abstr. 27:948); Bowen, W. D. et al. (2000) (“ACTIVATION OF SIGMA-2 RECEPTORS CAUSES CHANGES IN CERAMIDE LEVELS IN NEURONAL AND NON-NEURONAL CELL LINES,” Soc. Neurosci. Abstr. 26:601); and Crawford, K. W. et al. (2001) (“EVIDENCE FOR SIGMA-2 RECEPTOR CONTROL OF SPHINGOLIPID SIGNALING VIA SPHINGOLIPID CERAMIDE N-DEACYLASE (SCDASE) IN BREAST TUMOR CELLS,” PROC. AACR 42:3659).

### SUMMARY OF THE INVENTION

The ability of HIV to infect lymphocytes requires sphingomyelin, compounds that attenuate sphingomyelin production (or its presence in the cellular membranes) have the potential to inhibit or prevent immunodeficiency virus infection, especially human immunodeficiency virus (HIV) infection. The present invention particularly relates to sigma-2 receptor agonists and their use in the treatment of human immunodeficiency virus infections. The invention especially concerns sigma-2 agonists, especially CB-184 and its analogues, that decrease membrane sphingomyelin content when provided to recipient cells, and inhibit HIV replication and infection.

In detail, the invention concerns a method for treating actual or potential infection by an immunodeficiency virus in a mammal, wherein the method comprises administering to the mammal a therapeutically or prophylactically effective amount of a sigma-2 agonist.

The invention particularly concerns the embodiment of such method wherein the method is a method for treating actual infection by an immunodeficiency virus, and the administered amount of the sigma-2 agonist is a therapeutically effective amount.

The invention further concerns the embodiments of such methods wherein the method is a method for treating potential infection by an immunodeficiency virus, and the administered amount of the sigma-2 agonist is a prophylactically effective amount.

The invention particularly concerns the embodiments of such methods wherein the mammal is a human and the immunodeficiency virus is HIV, wherein the mammal is a simian and the immunodeficiency virus is SIV, and wherein the mammal is a feline and the immunodeficiency virus is a feline immunodeficiency virus.

The invention particularly concerns the embodiments of such methods wherein the sigma-2 agonist is a sigma-2 selective agonist, and especially wherein the sigma-2 agonist is a (+)-5,8-disubstituted morphan-7-one, or salt thereof. The invention particularly concerns the embodiments of such methods wherein the (+)-5,8-disubstituted morphan-7-one is CB-184 {(+)-1R,5R-E-8-(3,4-dichlorobenzylidene)-5-(3-hydroxyphenyl)-2-methylmorphan-7-one}.

The invention further provides the embodiment of the above-described methods wherein the method additionally comprises providing the mammal with a therapeutically effective amount of a drug that disrupts the life cycle of the virus, a drug that impairs the binding of the virus to a target cell, or a nucleotide analog.

The invention further provides the embodiment of the above-described methods wherein the method additionally comprises providing the mammal with a prophylactically effective amount of a drug that disrupts the life cycle of the virus, a drug that impairs the binding of the virus to a target cell, or a nucleotide analog.

The invention further provides an anti-immunodeficiency virus composition, the composition comprising an amount of a sigma-2 agonist effective for treating an actual or potential immunodeficiency virus infection in a mammal.

The invention particularly concerns the embodiment of such anti-immunodeficiency virus composition wherein the sigma-2 agonist is a sigma-2 selective agonist. The invention further concerns the embodiment of such anti-immunodeficiency virus composition wherein the sigma-2 agonist is a (+)-5,8-disubstituted morphan-7-one, or a salt thereof. The invention particularly concerns the embodiment of such anti-immunodeficiency virus composition wherein the (+)-5,8-disubstituted morphan-7-one is CB-184 {(+)-1R,5R-E-8-(3,4-dichlorobenzylidene)-5-(3-hydroxyphenyl)-2-methylmorphan-7-one}.

The invention additionally concerns the embodiments of the above-described anti-immunodeficiency virus compositions wherein the composition additionally comprises a drug that disrupts the life cycle of the virus, a drug that impairs the binding of the virus to a target cell, or a nucleotide analog. The invention particularly concerns the embodiment of such anti-immunodeficiency virus compositions wherein the virus is HIV, and the mammal is a human.

### BRIEF DESCRIPTION OF THE FIGURES

FIG. 1 shows the effect of sigma-2 agonists on sphingomyelin levels in H9 lymphocytes incubated in the presence of CB-184 or Haldol.

FIG. 2 shows the effect of CB184, CB64D, BD737 and haldol on HIV infection.

FIG. 3 shows the effect of compound AC-927 and CB-184 on LuSIV cell susceptibility to HIV infection using the lucerferase assay.

FIG. 4 shows the interaction between AC-927 and CB-184 on HIV infection.

FIG. 5 shows the interaction between compound AC-927 and CB-184 on HIV infection.

### DESCRIPTION OF THE PREFERRED EMBODIMENTS

The present invention derives in part from the discovery that compounds that activate cellular sigma-2 receptors are associated with a diminished presence of sphingomyelin in the cellular membrane. Although such compounds have generally been recognized as cytotoxic, one aspect of the present invention relates to the recognition that sub-cytotoxic doses of such compounds inhibit HIV replication. Notably, because such compounds do not target viral protein, the invention surmounts the capacity of HIV to mutate into forms that are resistant to anti-viral agents. Thus, the invention provides a potential treatment for blocking HIV infection that would overcome the most significant challenge encountered in the use of alternative HIV treatment regimes.

This invention involves the use of a sigma-2 agonist for treating patients suffering from HIV infection. As used herein, a “sigma-2 agonist” is a compound that activates cellular sigma-2 receptors. Any ligand with sigma-2 agonistic activity can be used in accordance with the present invention.

A preferred sigma-2 agonist is one which exhibits a greater binding preference for the sigma-2 receptor relative to the sigma-1 receptor and, thus, is defined as a “sigma-2 selective” agonist. More preferred is a sigma-2 selective agonist that exhibits about a 25-fold or greater binding preference for the sigma-2 receptor relative to the sigma-1 receptor. A still more preferred sigma-2 selective agonist exhibits about a 50-fold or greater selectivity for the sigma-2 receptor relative to the sigma-1 receptor, and a most preferred sigma-2 selective agonist exhibits about a 100-fold or greater selectivity for the sigma-2 receptor relative to the sigma-1 receptor.

Particularly preferred sigma-2 agonists are disclosed in U.S. Pat. Nos. 5,130,330, 5,346,908, 5,571,832, 5,571,832, 5,656,625, 5,656,625, 5,679,673, 5,679,679, 5,679,679, 5,739,158, 5,856,318 and 6,310,064. Thus, preferred compounds include compounds of the formula: 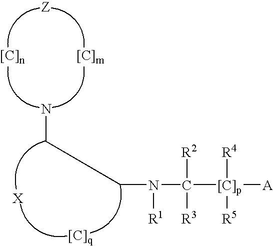
wherein R¹ is selected from hydrido, alkyl, cycloalkyl, hydroxyalkyl, haloalkyl, cycloalkylalkyl, alkoxyalkyl, aralkyl, aryl, alkenylalkyl, alkynylalkyl, carboxyalkyl, alkanoyl, alkylsulfinyl, alkylsulfonyl, arylsulfinyl and arylsulfonyl; wherein each of R² and R³ is independently selected from hydrido, alkyl, cycloalkyl, hydroxyalkyl, haloalkyl, cycloalkylalkyl, alkoxyalkyl, aralkyl, aryl, alkenyl, alkynyl, alkenylalkyl, alkynylalkyl, carboxyalkyl, alkanoyl, alkoxycarbonyl, carboxy, cyanoalkyl, alkylsulfinyl, alkylsulfonyl, arylsulfinyl and arylsulfonyl; wherein R² and R³ may be taken together to form a saturated or partially unsaturated carbocyclic group having three to eight ring carbons; wherein each of R⁴ through R¹¹ is independently selected from hydrido, hydroxy, alkyl, cycloalkyl, cycloalkylalkyl, aralkyl, aryl, alkoxy, aryloxy, aralkoxy, alkoxyalkyl, haloalkyl, hydroxyalkyl, cyano, amino, monoalkylamino, dialkylamino, carboxy, carboxyalkyl, alkanoyl, alkenyl and alkynyl; wherein R⁴ and R⁵ may be taken; together to form oxo or to form a saturated or partially unsaturated carbocyclic group having three to eight ring carbons; wherein R⁶ and R⁷ may be taken together to form oxo; wherein R⁸ and R⁹ may be taken together to form oxo; wherein R¹⁰ and R¹¹ may be taken together to form oxo; wherein each of n and m is a number selected from one through four; wherein each of p and q is a number selected from zero through five; wherein each of X and Z is independently selected from 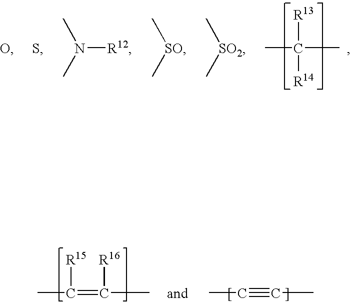
wherein R¹² may be selected from hydrido, alkyl, cycloalkyl, cycloalkylalkyl, aryl, aralkyl, heteroaryl, alkoxyalkyl, hydroxyalkyl, alkanoyl, aralkanoyl, aroyl, aminoalkyl, monoalkylaminoalkyl and dialkylaminoalkyl; wherein each of R¹³ through R¹⁶ is independently selected from hydrido, hydroxy, alkyl, cycloalkyl, cycloalkylalkyl, aralkyl, aryl, alkoxy, aralkoxy, aryloxy, alkoxyalkyl, haloalkyl, hydroxyalkyl, halo, cyano, amino, monoalkylamino, dialkylamino, carboxy, carboxyalkyl and alkanoyl; wherein A is selected from aryl, heteroaryl, aryloxy, heteroaryloxy, aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically-acceptable salt thereof.

A preferred family of such compounds consists of those compounds wherein R¹ is selected from hydrido, alkyl, cycloalkyl, hydroxyalkyl, haloalkyl, cycloalkylalkyl, alkoxyalkyl, aralkyl, aryl, alkenylalkyl, alkynylalkyl and carboxyalkyl; wherein each of R² and R³ is independently selected from hydrido, alkyl, cycloalkyl, hydroxyalkyl, haloalkyl, cycloalkylalkyl, alkoxyalkyl, aralkyl, aryl, alkenyl, alkynyl, alkenylalkyl, alkynylalkyl, carboxyalkyl, alkanoyl, alkoxycarbonyl, carboxy and cyanoalkyl; wherein R² and R³ may be taken together to form a saturated or partially unsaturated carbocyclic group having three to eight ring carbons; wherein each of R⁴ through R¹¹ is independently selected from hydrido, hydroxy, alkyl, cycloalkyl, cycloalkylalkyl, aralkyl, aryl, alkoxy, aryloxy, aralkoxy, alkoxyalkyl, haloalkyl, hydroxyalkyl, cyano, amino, monoalkylamino, dialkylamino, carboxy, carboxyalkyl, alkanoyl, alkenyl and alkynyl; wherein R⁴ and R⁵ may be taken together to form oxo or to form a saturated or partially unsaturated carbocyclic group having three to eight ring carbons; wherein each of n and m is a number selected from one through four; wherein each of p and q is a number selected from zero through five; wherein X is selected from 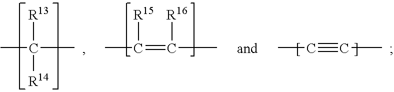
wherein Z is selected from O, 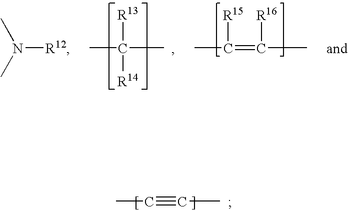
   wherein R¹² may be selected from hydrido, alkyl, cycloalkyl, cycloalkylalkyl, aryl, aralkyl, heteroaryl, alkoxyalkyl, hydroxyalkyl, alkanoyl, aralkanoyl, aroyl, aminoalkyl, monoalkylaminoalkyl and dialkylaminoalkyl; wherein each of R¹³ through R¹⁶ is independently selected from hydrido, hydroxy, alkyl, cycloalkyl, cycloalkylalkyl, aralkyl, aryl, alkoxy, aralkoxy, aryloxy, alkoxyalkyl, haloalkyl, hydroxyalkyl, halo, cyano, amino, monoalkylamino, dialkylamino, carboxy, carboxyalkyl and alkanoyl; wherein A is selected from aryl, heteroaryl, aryloxy, heteroaryloxy, aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically acceptable salt thereof.

A more preferred family of such compounds consists of those compounds wherein R¹ is selected from hydrido, lower alkyl, cycloalkyl of three to about eight carbon atoms, hydroxy-lower alkyl, halo-lower alkyl, cycloalkylalkyl of four to about eight carbon atoms, lower alkoxy-lower alkyl, phenyl-lower alkyl, phenyl, lower alkenyl-lower alkyl, lower alkynyl-lower alkyl and carboxy lower alkyl; wherein each of R² and R³ is independently selected from hydrido, lower alkyl, cycloalkyl of three to about eight carbon atoms, hydroxy lower alkyl, halo lower alkyl, cycloalkylalkyl of four to about eight carbon atoms, lower alkoxy lower alkyl, phenyl lower alkyl, phenyl, lower alkenyl, lower alkynyl, lower alkenyl lower alkyl, lower alkynyl lower alkyl, carboxy lower alkyl, lower alkanoyl, lower alkoxycarbonyl, carboxy and cyano lower alkyl; wherein R² and R³ may be taken together to form a saturated or partially unsaturated carbocyclic group having three to eight ring carbons; wherein each of R⁴ through R¹¹ is independently selected from hydrido, hydroxy, lower alkyl, cycloalkyl of three to about eight carbon atoms, cycloalkylalkyl of four to about eight carbon atoms, phenyl lower alkyl, phenyl, lower alkoxy, phenoxy, phenyl lower alkoxy, lower alkoxy lower alkyl, halo lower alkyl, hydroxy lower alkyl, cyano, amino, mono lower alkylamino, di lower alkylamino, carboxy, carboxy lower alkyl, lower alkanol, lower alkenyl and lower alkynyl; wherein R⁴ and R⁵ may be taken together to form oxo or to form a saturated or partially unsaturated carbocyclic group having three to eight ring carbons; wherein each of n and m is a number selected from one through four; wherein each of p and q is a number selected from zero through five; wherein X is selected from 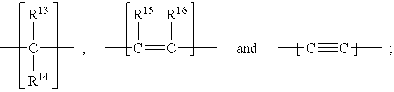
wherein Z is selected from O, 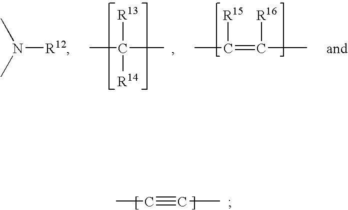
   wherein R¹² may be selected from hydrido, lower alkyl, cycloalkyl of three to about eight carbon atoms, cycloalkylalkyl of four to about eight carbon atoms, phenyl, phenyl lower alkyl, heteroaryl, lower alkoxy lower alkyl, hydroxy lower alkyl, lower alkanoyl, phenylalkanoyl, benzoyl, amino lower alkyl, mono lower alkylamino lower alkyl and di lower alkylamino lower alkyl; wherein each of R¹³ through R¹⁶ is independently selected from hydrido, hydroxy, lower alkyl, cycloalkyl of three to about eight carbon atoms, cycloalkylalkyl of four to about eight carbon atoms, pheny lower alkyl, phenyl, lower alkoxy, phenyl lower alkoxy, phenoxy, lower alkoxy lower alkyl, halo lower alkyl, hydroxy lower alkyl, halo, cyano, amino, mono lower alkylamino, di lower alkylamino, carboxy, carboxy lower alkyl and lower alkanoyl; wherein A is selected from phenyl, naphthyl, heteroaryl, phenoxy, naphthyloxy, heteroaryloxy, phenyl lower alkoxy, naphthyl lower alkoxy, heteroaryl lower alkoxy, phenylamino, naphthylamino, heteroarylamino, phenyl lower alkylamino, naphthyl lower alkylamino, heteroaralkylamino, phenylthio, naphthylthio, heteroarylthio, phenyl lower alkylthio and heteroaryl lower alkylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, phenyl lower alkyl, phenyl, lower alkoxy, phenoxy, pheny lower alkoxy, lower alkoxy lower alkyl, halo, halo lower alkyl, hydroxy lower alkyl, cyano, amino, mono lower alkylamino, di lower alkylamino, carboxy, carboxy lower alkyl, lower alkanoyl, lower alkenyl and lower alkynyl; or a pharmaceutically acceptable salt thereof.

A more highly preferred family of such compounds consists of those compounds wherein R¹ is selected from hydrido, lower alkyl, cycloalkyl of three to about eight carbon atoms, cycloalkylalkyl of four to about eight carbon atoms, benzyl, phenyl, lower alkenyl lower alkyl and lower alkynyl lower alkyl; wherein each of R² and R³ is independently selected from hydrido, lower alkyl, cycloalkyl of three to about eight carbon atoms, cycloalkylalkyl of four to about eight carbon atoms, benzyl, phenyl, lower alkenyl, lower alkynyl, lower alkenyl lower alkyl, lower alkynyl lower alkyl, lower alkanoyl and lower alkoxycarbonyl; wherein R² and R³ may be taken together to form a saturated or partially unsaturated carbocyclic group having three to eight ring carbons; wherein each of R⁴ through R¹¹ is independently selected from hydrido, hydroxy, lower alkyl, cycloalkyl of three to about eight carbon atoms, cycloalkylalkyl of four to about eight carbon atoms, benzyl, phenyl, lower alkoxy, phenoxy, benzyloxy, lower alkoxy lower alkyl, halo lower alkyl, hydroxy lower alkyl, lower alkanoyl, lower alkenyl and lower alkynyl; wherein R⁴ and R⁵ may be taken together to form oxo or to form a saturated or partially unsaturated carbocyclic group having three to eight ring carbons; wherein each of n and m is a number selected from one through four; wherein each of p and q is a number selected from zero through five; wherein X is selected from 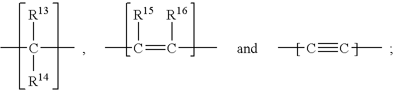
wherein Z is selected from O, 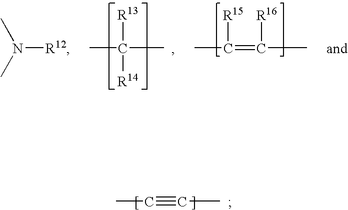
   wherein R¹² may be selected from hydrido, lower alkyl, cycloalkyl of three to about eight carbon atoms, cycloalkylalkyl of four to about eight carbon atoms, phenyl, benzyl, lower alkoxy lower alkyl and hydroxy lower alkyl; wherein each of R¹³ through R¹⁶ is independently selected from hydrido, hydroxy, lower alkyl, cycloalkyl of three to about eight carbon atoms, cycloalkylalkyl of four to about eight carbon atoms, benzyl, phenyl, lower alkoxy, benzyloxy, phenoxy, lower alkoxy lower alkyl, halo lower alkyl, hydroxy lower alkyl and halo; wherein A :is selected from phenyl, naphthyl, thienyl, phenoxy, benzyloxy, naphthyloxy, thiophenoxy, phenylamino, benzylamino, naphthylamino, phenylthio, benzylthio and naphthylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, lower alkoxy, lower alkoxy lower alkyl, halo, halo lower alkyl, hydroxy lower alkyl, amino, mono lower alkylamino, di lower alkylamino, lower alkanoyl, lower alkenyl and lower alkynyl; or a pharmaceutically acceptable salt thereof.

A family of such compounds of particular interest are compounds embraced by the formula: 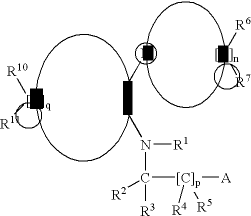
wherein R¹ is selected from hydrido, lower alkyl, cycloalkylalkyl of four to six carbon atoms and lower alkenyl lower alkyl; wherein each of R² and R³ is independently selected from hydrido and lower alkyl; wherein each of R⁴ through R⁷, R¹⁰ and R¹¹ is independently selected from hydrido, hydroxy, lower alkyl, benzyl, phenoxy, benzyloxy and halo lower alkyl; wherein n is a number selected from four through six; wherein p is a number selected from zero through four; wherein q is a number selected from three through five; wherein A is selected from phenyl, naphthyl and thienyl; wherein any of the foregoing A groups can be further substituted with one or more substituents independently selected from hydrido, hydroxy, lower alkyl, lower alkoxy, halo, halo lower alkyl, amino, mono lower alkylamino and di lower alkylamino; or a pharmaceutically acceptable salt thereof.

A more preferred family of such compounds consists of compounds wherein R¹ is selected from hydrido, methyl, ethyl, propyl, cyclopropylmethyl, allyl and dimethylallyl; wherein each of R² and R³ is independently selected from hydrido, methyl, ethyl and propyl; wherein each of R⁴ through R⁷, R¹⁰ and R¹¹ is independently selected from hydrido, hydroxy, methyl, ethyl, propyl, benzyl, phenoxy, benzyloxy and halo lower alkyl; wherein n is a number selected from four or five; wherein p is a number selected from zero through two; wherein q is a number selected from three or four; wherein A is phenyl or naphthyl; wherein any of the foregoing A groups can be further substituted with one or more substituents independently selected from hydroxy, methyl, ethyl, propyl, methoxy, ethoxy, methylenedioxy, halo, trifluoromethyl, amino, methylamino and dimethylamine; or a pharmaceutically acceptable salt thereof.

Compounds of the following formula are particularly preferred: 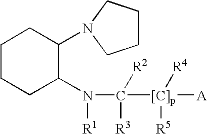
wherein R¹ is selected from the group consisting of hydrogen, C₁-C₅ alkyl, C₃-C₈ cycloalkyl or C₄-C₈ cycloalkylalkyl with the proviso that when R1 is methyl, than p is not 1; R², R³, R⁴ and R⁵ are hydrogen; and A is phenyl and wherein A may be further substituted with one or more halo substituents.

Particularly preferred examples of such compounds include:
(±)-cis-N-[2-(3,4-dichlorophenyl)ethyl]-2-(1-pyrrolidinyl)-cyclohexylamine;
1S,2R-(+)-cis-N-[2-(3,4-dichlorophenyl)ethyl]-2-(1-pyrrolidinyl)-cyclohexylamine;
1R,2S-(−)-cis-N-[2-(3,4-dichlorophenyl)ethyl]-2-(1-pyrrolidinyl)-cyclohexylamine;
1S,2R-(+)-cis-N-[2-(3,4-dichlorophenyl)ethyl]-N-ethyl-2-(1-pyrrolidinyl)cyclohexylamine;
1R,2S(−)-cis-N-[2-(3,4-dichlorophenyl) ethyl]N-ethyl-2-(1-pyrrolidinyl)cyclohexylamine;
1S,2R-(+)-cis-N-[2-(3,4-dichlorophenyl)ethyl]-N-(1-propyl)-2-(1-pyrrolidinyl)cyclohexylamine;
1R,2S-(−)-cis-N-[2-(3,4-dichlorophenyl)ethyl]-N-(1-propyl)-2-(1-pyrrolidinyl)cyclohexylamine;
1S,2R-(+)-cis-N-[2-(3,4,-dichlorophenyl)ethyl]-N-cyclopropylmethyl-1-(1-pyrrolidinyl)cyclohexylamine;
1R,2S-(−)-cis-N-[2-(3,4-dichlorophenyl)ethyl]-N-cyclopropylmethyl-2-(1-pyrrolidinyl)-cyclohexylamine; and
(±)-cis-N-[2-(3,4-dichlorophenyl)methyl]-N-methyl-2-(1-pyrrolidinyl)-cyclohexylamine;

BD737 is a particularly preferred sigma-2 agonist. BD737 is 1S,2R-(+)-cis-N-[2-(3,4-dichlorophenyl)ethyl]-N-methyl-2-(1-pyrrolidinyl)-cyclohexylamine (Bowen, W. D. et al. (1992) “CHARACTERIZATION OF THE ENANTIOMERS OF CIS-N-[2-(3,4-DICHLOROPHENYL)ETHYL]-N-METHYL-2-(1-PYRROLIDINYL) CYCLOHEXYLAMINE (BD737 AND BD738): NOVEL COMPOUNDS WITH HIGH AFFINITY, SELECTIVITY, AND BIOLOGICAL EFFICACY AT SIGMA RECEPTORS,” J. Pharmacol. Exp. Ther. 262:32-40; Vilner, B. J. et al. (2000) “MODULATION OF CELLULAR CALCIUM BY SIGMA-2 RECEPTORS: RELEASE FROM INTRACELLULAR STORES IN HUMAN SK-N-SH NEUROBLASTOMA CELLS,” J. Pharmacol. Exp. Ther. 292(3):900-11).

Additional examples of sigma-2 agonists include compounds of the formula: 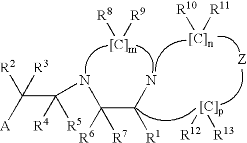
wherein each of R¹, R⁴, R⁵, R⁶ and R⁷ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein each of R², R3 and R⁸ through R¹³ is independently selected from hydrido, hydroxy, alkyl, cycloalkyl, cycloalkylalkyl, aralkyl, aryl, alkoxy, aryloxy, aralkoxy, alkoxyalkyl, haloalkyl, hydroxyalkyl, cyano, amino, monoalkylamino, dialkylamino, carboxy, carboxyalkyl, alkanoyl, alkenyl and alkynyl; wherein R² and R³ may be taken together to form oxo or to form a saturated or partially unsaturated carbocyclic group having three to eight ring carbons; wherein R⁴ and R⁵ may be taken together to form a saturated or partially unsaturated carbocyclic group having three to eight Ting carbons; wherein R⁸ and R⁹ may be taken together to form oxo; wherein R¹⁰ and R¹¹ may be taken together to form oxo; wherein m is an integer from 2-4 and n and p are integers of from one to four; wherein Z is selected from 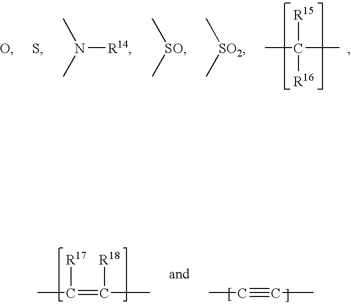
wherein R¹⁴ may be selected from hydrido, alkyl, cycloalkyl, cycloalkylalkyl, aryl, aralkyl, heteroaryl, alkanoyl aralkanoyl, aroyl, aminoalkyl, monoalkylaminoalkyl and dialkylaminoalkyl; wherein each of R¹⁵ and R¹⁶ is independently selected from hydrido, hydroxy, alkyl, cycloalkyl, cycloalkylalkyl, aralkyl, aryl, alkoxy, aralkoxy, aryloxy, alkoxyalkyl, haloalkyl, hydroxyalkyl, halo, cyano, amino, monoalkylamino, dialkylamino, carboxy, carboxyalkyl and alkanoyl; wherein each of R¹⁷ and R¹⁸ is independently selected from hydrido, alkyl, cycloalkyl, cycloalkylalkyl, aralkyl, alkoxyalkyl, haloalkyl, hydroxyalkyl, halo, cyano, carboxy, carboxyalkyl and alkanoyl; wherein A is selected from aryl, heteroaryl, aryloxy, heteroaryloxyl aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically-acceptable salt thereof.

A preferred family of such compounds consists of those compounds wherein each of R¹ R⁴, R⁵, R⁶ and R⁷ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein each of R², R³ and R⁸ through R¹³ is independently selected from hydrido, hydroxy, alkyl, cycloalkyl, cycloalkylalkyl, aralkyl, aryl, alkoxy, aryloxy, aralkoxy, alkoxyalkyl, haloalkyl, hydroxyalkyl, cyano, amino, monoalkylamino, dialkylamino, carboxy, carboxyalkyl, alkanoyl, alkenyl and alkynyl; wherein R² and R³ may be taken together to form oxo or to form a saturated or partially unsaturated carbocyclic group having three to eight ring carbons; wherein R⁴ and R⁵ may be taken together to form oxo or to form a saturated or partially unsaturated carbocyclic group having three to eight ring carbons; wherein R¹⁰ and R¹¹ may be taken together to form oxo; wherein m is an integer from 2-4 and n and p are integers of from one to four; wherein Z is selected from 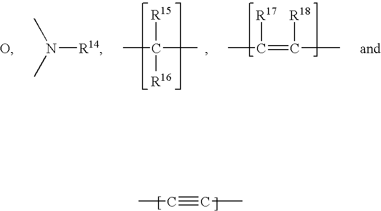
wherein R¹⁴ may be selected from hydrido, alkyl, cycloalkyl, cycloalkylalkyl, aryl, aralkyl, heteroaryl, alkanoyl, aralkanoyl and aroyl; wherein each of R¹⁵ through R¹⁸ is independently selected from hydrido, alkyl, cycloalkyl, cycloalkylalkyl, aralkyl, aryl, alkoxyalkyl, haloalkyl, hydroxyalkyl, halo, cyano, carboxy, carboxyalkyl and alkanoyl; wherein A is selected from aryl, heteroaryl, aryloxy, heteroaryloxy, aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically acceptable salt thereof.

A more preferred family of such compounds consists of those compounds wherein each of R¹, R⁴, R⁵, R⁶ and R⁷ is independently selected from hydrido, loweralkyl, cycloalkyl of three to about eight carbon atoms, phenylloweralkyl, phenyl, loweralkoxyloweralkyl, haloloweralkyl; hydroxyloweralkyl, carboxy, carboxyloweralkyl, loweralkanyl, loweralkenyl, loweralkynyl; wherein R², R³ and R⁸ through R¹³ is independently selected from hydrido, hydroxy, loweralkyl, cycloalkyl of three to about eight carbon atoms, cycloalkylalkyl of four to about eight carbon atoms, phenylloweralkyl, phenyl, loweralkoxy, phenoxy, phenylloweralkoxy, loweralkoxyloweralkyl, haloloweralkyl, hydroxyloweralkyl, cyano, amino, monoloweralkylamino, diloweralkylamino, carboxy, carboxyloweralkyl, loweralkanoyl, loweralkenyl and loweralkynyl; wherein R² and R³ may be taken together to form a saturated or partially unsaturated carbocyclic group having three to eight ring carbons; wherein R⁴ and R⁵ may be taken together to form a saturated or partially unsaturated carbocyclic group having three to eight ring carbons; wherein R¹⁰ and R¹¹ may be taken together to form oxo; wherein m is an integer from 2-4 and n and p are integers of from one to four; wherein Z is selected from 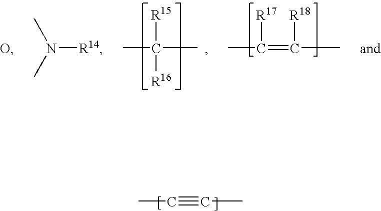
wherein R¹⁴ may be selected from hydrido, lower alkyl, cycloalkyl of three to about eight carbon atoms, cycloalkylalkyl of four to about eight carbon atoms, phenyl, phenyl lower alkyl, heteroaryl, lower alkanoyl, phenylalkanoyl, benzoyl, amino lower alkyl, mono lower alkyl-amino lower alkyl and di lower alkylamino-lower alkyl; wherein each of R¹⁵ and R¹⁶ is independently selected from hydrido, hydroxy, lower alkyl, cycloalkyl of three to about eight carbon atoms, cycloalkylalkyl of four to about eight carbon atoms, pheny lower alkyl, phenyl, lower alkoxy lower alkyl, halo lower alkyl, hydroxy lower alkyl, halo, cyano, amino, mono lower alkylamino, di lower alkylamino, carboxy, carboxy lower alkyl and lower alkanoyl; wherein each of R¹⁷ and R¹⁸ is independently selected from hydrido, lower alkyl, cycloalkyl of three to about eight carbon atoms, cycloalkylalkyl of four to about eight carbon atoms, phenyl lower alkyl, phenyl, lower alkoxy lower alkyl, halo lower alkyl, hydroxy lower alkyl, halo, cyano, carboxy, carboxy lower alkyl and lower alkanoyl; wherein A is selected from phenyl, naphthyl, heteroaryl, phenoxy, naphthyloxy, heteroaryloxy, phenyl lower alkoxy, naphthyl lower alkoxy, heteroaryl lower alkoxy, phenylamino, naphthylamino, heteroarylamino, phenyl lower alkylamino, naphthyl lower alkylamino, heteroaralkylamino, phenylthio, naphthylthio, heteroarylthio, phenyl lower alkylthio and heteroaryl lower alkylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, phenyl lower alkyl, phenyl, lower alkoxy, phenoxy, pheny lower alkoxy, lower alkoxy lower alkyl, halo, halo lower alkyl, hydroxy lower alkyl, cyano, amino, mono lower alkylamino, di lower alkylamino, carboxy, carboxy lower alkyl, lower alkanoyl, lower alkenyl and lower alkynyl; or a pharmaceutically acceptable salt thereof.

A more highly preferred family of such compounds consists of those compounds wherein each of R¹, R⁴, R⁵, R⁶ and R⁷ is independently selected from hydrido, lower alkyl, cycloalkyl of three to about eight carbon atoms, cycloalkylalkyl of four to about eight carbon atoms, benzyl, phenyl, lower alkoxy lower alkyl, halo lower alkyl, hydroxy lower alkyl, lower alkanoyl, lower alkenyl, and lower alkynyl; wherein R², R³ and R⁸ through R¹³ is independently selected from hydrido, hydroxy, lower alkyl, cycloalkyl of three to about eight carbon atoms, cycloalkylalkyl of four to about eight carbon atoms, benzyl, phenyl, lower alkoxy, phenoxy, benzyloxy, lower alkoxy lower alkyl, halo lower alkyl, hydroxy lower alkyl, lower alkanoyl, lower alkenyl and lower alkynyl; wherein R² and R³ may be taken together to form a saturated or partially unsaturated carbocyclic group having three to eight ring carbons; wherein R⁴ and R⁵ may be taken together to form a saturated or partially unsaturated carbocyclic group having three to eight ring carbons; wherein R¹⁰ and R¹¹ may be taken together to form oxo; wherein m is an integer from 2-4 and n and p are integers of from one to four; wherein Z is selected from 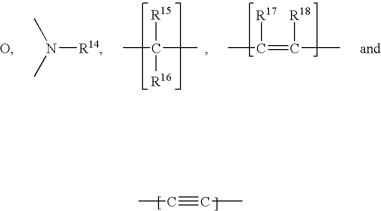
wherein R¹⁴ may be selected from hydrido, lower alkyl, cycloalkyl of three to about eight carbon atoms, cycloalkylalkyl of four to about eight carbon atoms, phenyl and benzyl; wherein each of R¹⁵ through R¹⁸ is independently selected from hydrido, lower alkyl, cycloalkyl of three to about eight carbon atoms, cycloalkylalkyl of four to about eight carbon atoms, benzyl, phenyl, lower alkoxy lower alkyl, hydroxy lower alkyl and halo; wherein A is selected from phenyl, naphthyl, benzo[b]thienyl, thienyl, phenoxy, benzyloxy, naphthyloxy, thiophenoxy, phenylamino, benzylamino, naphthylamino, phenylthio, benzylthio and naphthylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, lower alkoxy, lower alkoxy lower alkyl, halo, halo lower alkyl, hydroxy lower alkyl, amino, mono lower alkylamino, di lower alkylamino, lower alkanoyl, lower alkenyl and lower alkynyl; or a pharmaceutically acceptable salt thereof.

A family of compounds of particular interest within Formula I are compounds embraced by the formula: 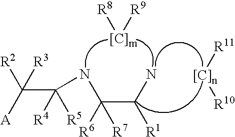
wherein each of R¹, R⁴, R⁵, R⁶ and R⁷ is independently selected from hydrido, lower alkyl, benzyl and halo lower alkyl; wherein R², R³ and R⁸ through R¹¹ is independently selected from hydrido, hydroxy, lower alkyl, benzyl, phenoxy, benzyloxy and halo lower alkyl; wherein n is an integer of from four to six; wherein m is an integer of from two to four; wherein A is selected from phenyl, naphthyl, benzothienyl, benzofuranyl and thienyl; wherein any of the foregoing A groups can be further substituted with one or more substituents independently selected from hydrido, hydroxy, lower alkyl, lower alkoxy, halo, halo lower alkyl, amino, mono lower alkylamino and di lower alkylamino; or a pharmaceutically acceptable salt thereof.

A more preferred family of such compounds consists of compounds wherein each of R¹, R⁴, R⁵, R⁶ and R⁷ is independently selected from hydrido, methyl, ethyl, propyl, benzyl, and halo lower alkyl, wherein R², R³ and R⁸ through R¹¹ is independently selected from hydrido, hydroxy, methyl, ethyl, propyl, benzyl, phenoxy, benzyloxy and halo lower alkyl; wherein n is a number selected from 4 or 5; wherein in is an integer of from two or three; wherein A is phenyl or naphthyl; wherein any of the foregoing A groups can be further substituted with one or more substituents independently selected from hydroxy, methyl, ethyl, propyl, methoxy, ethoxy, methylenedioxy, halo, trifluoromethyl, amino, methylamino and dimethylamino; or a pharmaceutically acceptable salt thereof.

The following specific compounds are particularly preferred:
2-[2-(3,4-dichlorophenyl)ethyl]-2,5-diazabicyclo[3.0.4]nonane
2-[2-(3-benzothienyl)ethyl]-2,5-diazabicyclo[3.0.4]nonane
2-[2-naphthylethyl]-2,5-diazabicyclo[3.0.4]nonane
2-[2-(3,4-dichlorophenyl)ethyl]-2,5-diazabicyclo[4.0.4]decane
2-[2-(3-benzothienyl)ethyl]-2,5-diazabicyclo[4.0.4]decane
2-[2-naphthylethyl]-2,5-diazabicyclo[4.0.4]decane

Additional examples of sigma-2 agonists include compounds of the formula: 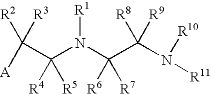
wherein each of R¹, R¹⁰ and R¹¹ is selected from hydrido, alkyl, cycloalkyl, hydroxyalkyl, haloalkyl, cycloalkylalkyl, alkoxyalkyl, aralkyl, aryl, alkenylalkyl, alkynylalkyl, carboxyalkyl, alkanoyl, alkylsulfinyl, alkylsulfonyl, arylsulfinyl and arylsulfonyl; wherein each of R² and R³ is independently selected from hydrido, alkyl, cycloalkyl, hydroxyalkyl, haloalkyl, cycloalkylalkyl, alkoxyalkyl, aralkyl, aryl, alkenyl, alkynyl, alkenylalkyl, alkynylalkyl, carboxyalkyl, alkanoyl, alkoxycarbonyl, carboxy, cyanoalkyl, alkylsulfinyl, alkylsulfonyl, arylsulfinyl and arylsulfonyl; wherein each of R⁴ through R⁹ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein R² and R³ may be taken together to form oxo; wherein R⁴ and R⁵ may be taken together to form oxo; wherein R⁶ and R⁷ may be taken together to form oxo; wherein R⁸ and R⁹ may be taken together to form oxo; wherein one of R¹⁰ or R¹¹ together with one of R⁶ or R⁷, or together with one of R⁸ or R⁹, form a heterocyclic ring moiety which includes the nitrogen atom to which R¹⁰ and R¹¹ are attached, said ring moiety having from five to ten ring members; wherein A is selected from aryl, heteroaryl, aryloxy, heteroaryloxy, aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically-acceptable salt thereof.

A preferred family of such compounds consists of those compounds wherein each of R¹, R¹⁰ and R¹¹ is selected from hydrido, alkyl, cycloalkyl, hydroxyalkyl, haloalkyl, cycloalkylalkyl, alkoxyalkyl, aralkyl, aryl, alkenylalkyl, alkynylalkyl and carboxyalkyl; wherein each of R² and R³ is independently selected from hydrido, alkyl, cycloalkyl, hydroxyalkyl, haloalkyl, cycloalkylalkyl, alkoxyalkyl, aralkyl, aryl, alkenyl, alkynyl, alkenylalkyl, alkynylalkyl, carboxyalkyl, alkanoyl, alkoxycarbonyl, carboxy and cyanoalkyl; wherein each of R⁴ through R⁹ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein R⁴ and R⁵ may be taken together to form oxo; wherein R⁶ and R⁷ may be taken together to form oxo; wherein R⁸ and R⁹ may be taken together to form oxo; wherein one of R¹⁰ and R¹¹ together with one of R⁶ and R⁷, or together with one of R⁸ and R⁹, form a heterocyclic ring moiety which includes the nitrogen atom to which R¹⁰ and R¹¹ are attached, said ring moiety having from five to ten ring members; wherein A is selected from aryl, heteroaryl, aryloxy, heteroaryloxy, aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically acceptable salt thereof.

A more preferred family of such compounds consists of those compounds wherein each of R¹, R¹⁰ and R¹¹ is selected from hydrido, lower alkyl, cycloalkyl of three to about eight carbon atoms, hydroxy lower alkyl, halo lower alkyl, cycloalkylalkyl of four to about eight carbon atoms, lower alkoxy lower alkyl, phenyl lower alkyl, phenyl, lower alkenyl lower alkyl, lower alkynyl lower alkyl and carboxy lower alkyl; wherein each of R² and R³ is independently selected from hydrido, lower alkyl, cycloalkyl of three to about eight carbon atoms, hydroxy lower alkyl, halo lower alkyl, cycloalkylalkyl of four to about eight carbon atoms, lower alkoxy lower alkyl, phenyl lower alkyl, phenyl, lower alkenyl, lower alkynyl, lower alkenyl lower alkyl, lower alkynyl lower alkyl, carboxy lower alkyl, lower alkanoyl, lower alkoxycarbonyl, carboxy and cyano lower alkyl; wherein each of R⁴ through R⁹ is independently selected from hydrido, lower alkyl, cycloalkyl of three to about eight carbon atoms, cycloalkylalkyl of four to about eight carbon atoms, phenyl lower alkyl, phenyl, lower alkoxy lower alkyl, halo lower alkyl, hydroxy lower alkyl, carboxy, carboxy lower alkyl, lower alkanoyl, lower alkenyl and lower alkynyl; wherein R⁴ and R⁵ may be taken together to form oxo; wherein R⁶ and R⁷ may be taken together to form oxo; wherein R⁸ and R⁹ may be taken together to form oxo; wherein one of R¹⁰ and R¹¹ together with one of R⁶ and R⁷, or together with one of R⁸ and R⁹, form a heterocyclic ring moiety which includes the nitrogen atom to which R¹⁰ and R¹¹ are attached, said ring moiety having from five to ten ring members; wherein A is selected from phenyl, naphthyl, heteroaryl, phenoxy, naphthyloxy, heteroaryloxy, phenyl lower alkoxy, naphthyl lower alkoxy, heteroaryl lower alkoxy, phenylamino, naphthylamino, heteroarylamino, phenyl lower alkylamino, natphthyl lower alkylamino, heteroaralkylamino, phenylthio, naphthylthio, heteroarylthio, phenyl lower alkylthio and heteroaryl lower alkylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, phenyl lower alkyl, phenyl, lower alkoxy, phenoxy, pheny lower alkoxy, lower alkoxy lower alkyl, halo, halo lower alkyl, hydroxy lower alkyl, cyano, amino, mono lower alkylamino, di lower alkylamino, carboxy, carboxy lower alkyl, lower alkanoyl, lower alkenyl and lower alkynyl; or a pharmaceutically acceptable salt thereof.

A more highly preferred family of such compounds consists of those compounds wherein each of R¹, R¹⁰ and R¹¹ is selected from hydrido, lower alkyl, cycloalkyl of three to about eight carbon atoms, cycloalkylalkyl of four to about eight carbon atoms, benzyl, phenyl, lower alkenyl lower alkyl and lower alkynyl lower alkyl; wherein each of R² and R³ is independently selected from hydrido, lower alkyl, cycloalkyl of three to about eight carbon atoms, cycloalkylalkyl of four to about eight carbon atoms, benzyl, phenyl, lower alkenyl, lower alkynyl, lower alkenyl lower alkyl, lower alkynyl lower alkyl, lower alkanoyl and lower alkoxycarbonyl; wherein each of R⁴ through R⁹ is independently selected from hydrido, lower alkyl, cycloalkyl of three to about eight carbon atoms, cycloalkylalkyl of four to about eight carbon atoms, benzyl, phenyl, lower alkoxy lower alkyl, halo lower alkyl, hydroxy lower alkyl, lower alkanoyl, lower alkenyl and lower alkynyl; wherein R⁴ and R⁵ may be taken together to form oxo; wherein R⁶ and R⁷ may be taken together to form oxo; wherein R⁸ and R⁹ may be taken together to form oxo; wherein one of R¹⁰ and R¹¹ together with one of R⁶ and R⁷, or together with one of R⁸ and R⁹, form a heterocyclic ring moiety which includes the nitrogen atom to which R¹⁰ and R¹¹ are attached, said ring moiety having from five to ten ring members; wherein A is selected from phenyl, naphthyl, thienyl, phenoxy, benzyloxy, naphthyloxy, thiophenoxy, phenylamino, benzylamino, naphthylamino, phenylthio, benzylthio and naphthylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, lower alkoxy, lower alkoxy lower alkyl, halo, halo lower alkyl, hydroxy lower alkyl, amino, mono lower alkylamino, di lower alkylamino, lower alkanoyl, lower alkenyl and lower alkynyl; or a pharmaceutically acceptable salt thereof.

A preferred sub-class of such compounds are compounds represented by the formula: 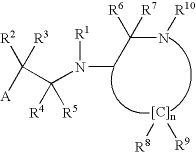
wherein each of R¹ and R¹⁰ is selected from hydrido, alkyl, cycloalkyl, hydroxyalkyl, haloalkyl, cycloalkylalkyl, alkoxyalkyl, aralkyl, aryl, alkenylalkyl, alkynylalkyl, carboxyalkyl, alkanoyl, alkylsulfinyl, alkylsulfonyl, arylsulfinyl and arylsulfonyl; wherein each of R² and R³ is independently selected from hydrido, alkyl, cycloalkyl, hydroxyalkyl, haloalkyl, cycloalkylalkyl, alkoxyalkyl, aralkyl, aryl, alkenyl, alkynyl, alkenylalkyl, alkynylalkyl, carboxyalkyl, alkanoyl, alkoxycarbonyl, carboxy, cyanoalkyl, alkylsulfinyl, alkylsulfonyl, arylsulfinyl and arylsulfonyl; wherein each of R⁴ through R⁹ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein R² and R³ may be taken together to form oxo; wherein R⁴ and R⁵ may be taken together to form oxo; wherein R⁶ and R⁷ may be taken together to form oxo; wherein R⁸ and R⁹ may be taken together to form oxo; wherein n is an integer of from 2 to 7; wherein A is selected from aryl, heteroaryl, aryloxy, heteroaryloxy, aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically-acceptable salt thereof.

A preferred family of such compounds are those compounds wherein R¹ and R¹⁰ is selected from hydrido, alkyl, cycloalkyl, hydroxyalkyl, haloalkyl, cycloalkylalkyl, alkoxyalkyl, aralkyl, aryl, alkenylalkyl, alkynylalkyl and carboxyalkyl; wherein each of R² and R³ is independently selected from hydrido, alkyl, cycloalkyl, hydroxyalkyl, haloalkyl, cycloalkylalkyl, alkoxyalkyl, aralkyl, aryl, alkenyl, alkynyl, alkenylalkyl, alkynylalkyl, carboxyalkyl, alkanoyl, alkoxycarbonyl, carboxy and cyanoalkyl; wherein each of R⁴ through R⁹ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein R⁴ and R⁵ may be taken together to form oxo; wherein R⁶ and R⁷ may be taken together to form oxo; wherein R⁸ and R⁹ may be taken together to form oxo; wherein A is selected from aryl, heteroaryl, aryloxy, heteroaryloxy, aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically acceptable salt thereof.

A more preferred family of such compounds are those compounds wherein R¹ and R¹⁰ is selected from hydrido, lower alkyl, cycloalkyl of three to about eight carbon atoms, hydroxy lower alkyl, halo lower alkyl, cycloalkylalkyl of four to about eight carbon atoms, lower alkoxy lower alkyl, phenyl lower alkyl, phenyl, lower alkenyl lower alkyl, lower alkynyl lower alkyl and carboxy lower alkyl; wherein each of R² and R³ is independently selected from hydrido, lower alkyl, cycloalkyl of three to about eight carbon atoms, hydroxy lower alkyl, halo lower alkyl, cycloalkylalkyl of four to about eight carbon atoms, lower alkoxy lower alkyl, phenyl lower alkyl, phenyl, lower alkenyl, lower alkynyl, lower alkenyl lower alkyl, lower alkynyl lower alkyl, carboxy lower alkyl, lower alkanoyl, lower alkoxycarbonyl, carboxy and cyano lower alkyl; wherein each of R⁴ through R⁹ is independently selected from hydrido, lower alkyl, cycloalkyl of three to about eight carbon atoms, cycloalkylalkyl of four to about eight carbon atoms, phenyl lower alkyl, phenyl, lower alkoxy lower alkyl, halo lower alkyl, hydroxy lower alkyl, carboxy, carboxy lower alkyl, lower alkanoyl, lower alkenyl and lower alkynyl; wherein R⁴ and R⁵ may be taken together to form oxo; wherein R⁶ and R⁷ may be taken together to form oxo; wherein R⁸ and R⁹ may be taken together to form oxo; wherein A is selected from phenyl, naphthyl, heteroaryl, phenoxy, naphthyloxy, heteroaryloxy, phenyl lower alkoxy, naphthyl lower alkoxy, heteroaryl lower alkoxy, phenylamino, naphthylamino, heteroarylamino, phenyl lower alkylamino, naphthyl lower alkylamino, heteroaralkylamino, phenylthio, naphthylthio, heteroarylthio, phenyl lower alkylthio and heteroaryl lower alkylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, phenyl lower alkyl, phenyl, lower alkoxy, phenoxy, pheny lower alkoxy, lower alkoxy lower alkyl, halo, halo lower alkyl, hydroxy lower alkyl, cyano, amino, mono lower alkylamino, di lower alkylamino, carboxy, carboxy lower alkyl, lower alkanoyl, lower alkenyl and lower alkynyl; or a pharmaceutically acceptable salt thereof.

A more highly preferred family of such compounds are those compounds wherein R¹ and R¹⁰ is selected from hydrido, lower alkyl, cycloalkyl of three to about eight carbon atoms, cycloalkylalkyl of four to about eight carbon atoms, benzyl, phenyl, lower alkenyl lower alkyl and lower alkynyl lower alkyl; wherein each of R² and R³ is independently selected from hydrido, lower alkyl, cycloalkyl of three to about eight carbon atoms, cycloalkylalkyl of four to about eight carbon atoms, benzyl, phenyl, lower alkenyl, lower alkynyl, lower alkenyl lower alkyl, lower alkynyl lower alkyl, lower alkanoyl and lower alkoxycarbonyl; wherein each of R⁴ through R⁹ is independently selected from hydrido, lower alkyl, cycloalkyl of three to about eight carbon atoms, cycloalkylalkyl of four to about eight carbon atoms, benzyl, phenyl, lower alkoxy lower alkyl, halo lower alkyl, hydroxy lower alkyl, lower alkanoyl, lower alkenyl and lower alkynyl; wherein R⁴ and R⁵ may be taken together to form oxo; wherein R⁶ and R⁷ may be taken together to form oxo; wherein R⁸ and R⁹ may be taken together to form oxo; wherein A is selected from phenyl, naphthyl, thienyl, phenoxy, benzyloxy, naphthyloxy, thiophenoxy, phenylamino, benzylamino, naphthylamino, phenylthio, benzylthio and naphthylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, lower alkoxy, lower alkoxy lower alkyl, halo, halo lower alkyl, hydroxy lower alkyl, amino, mono lower alkylamino, di lower alkylamino, lower alkanoyl, lower alkenyl and lower alkynyl; or a pharmaceutically acceptable salt thereof. Compounds of particular interest that fall within the scope of such formula include:
3-[N-(3,4-dichlorophenyl)ethyl]-N-(methyl)amino]-1-(methyl)piperidine
3-[N-(3,4-dichlorophenyl)ethyl]-N-(methyl)amino]-1-(methyl)homopiperidine
3-[N-(3,4-dichlorophenyl)ethyl]-N-(methyl)amino]-1-(methyl)pyrrolidine
3-[N-(3-benzothienyl)ethyl]-N-(methyl)amino]-1-(methyl)piperidine
3-[N-(3-benzothienyl)ethyl]-N-(methyl)amino]-1-(methyl)homopiperidine
3-[N-(3-benzothienyl)ethyl]-N-(methyl)amino]-1-(methyl)pyrrolidine
3-[N-(2-[2-napthyl]ethyl)]-N-(methyl)amino]-1-(methyl)piperidine
3-[N-(2-[2-napthyl]ethyl)]-N-(methyl)amino]-]-(methyl)homopiperidine
3-[N-(2-[2-napthyl]ethyl)]-N-(methyl)amino]-1-(methyl)pyrrol idine

Another preferred sub-class of compounds are those compounds represented by the formula: 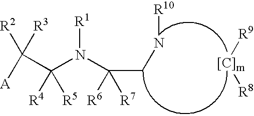
wherein each of R¹ and R¹⁰ is selected from hydrido, alkyl, cycloalkyl, hydroxyalkyl, haloalkyl, cycloalkylalkyl, alkoxyalkyl, aralkyl, aryl, alkenylalkyl, alkynylalkyl, carboxyalkyl, alkanoyl, alkylsulfinyl, alkylsulfonyl, arylsulfinyl and arylsulfonyl; wherein each of R² and R³ is independently selected from hydrido, alkyl, cycloalkyl, hydroxyalkyl, haloalkyl, cycloalkylalkyl, alkoxyalkyl, aralkyl, aryl, alkenyl, alkynyl, alkenylalkyl, alkynylalkyl, carboxyalkyl, alkanoyl, alkoxycarbonyl, carboxy, cyanoalkyl, alkylsulfinyl, alkylsulfonyl, arylsulfinyl and arylsulfonyl; wherein each of R⁴ through R⁹ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein R² and R³ may be taken together to form oxo; wherein R⁴ and R⁵ may be taken together to form oxo; wherein R⁶ and R⁷ may be taken together to form oxo; wherein R⁸ and R⁹ may be taken together to form oxo; wherein m is an integer of from 3 to 8; wherein A is selected from aryl, heteroaryl, aryloxy, heteroaryloxy, aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically-acceptable salt thereof.

A preferred family of such compounds are those compounds wherein each of R¹ and R¹⁰ is selected from hydrido, alkyl, cycloalkyl, hydroxyalkyl, haloalkyl, cycloalkylalkyl, alkoxyalkyl, aralkyl, aryl, alkenylalkyl, alkynylalkyl and carboxyalkyl; wherein each of R² and R³ is independently selected from hydrido, alkyl, cycloalkyl, hydroxyalkyl, haloalkyl, cycloalkylalkyl, alkoxyalkyl, aralkyl, aryl, alkenyl, alkynyl, alkenylalkyl, alkynylalkyl, carboxyalkyl, alkanoyl, alkoxycarbonyl, carboxy and cyanoalkyl; wherein each of R⁴ through R¹¹ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein R⁴ and R⁵ may be taken together to form oxo; wherein R⁶ and R⁷ may be taken together to form oxo; wherein R⁸ and R⁹ may be taken together to form oxo; wherein A is selected from aryl, heteroaryl, aryloxy, heteroaryloxy, aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically acceptable salt thereof.

A more preferred family of such compounds are those compounds wherein each of R¹ and R¹⁰ is selected from hydrido, lower alkyl, cycloalkyl of three to about eight carbon atoms, hydroxy lower alkyl, halo lower alkyl, cycloalkylalkyl of four to about eight carbon atoms, lower alkoxy lower alkyl, phenyl lower alkyl, phenyl, lower alkenyl lower alkyl, lower alkynyl lower alkyl and carboxy lower alkyl; wherein each of R² and R³ is independently selected from hydrido, lower alkyl, cycloalkyl of three to about eight carbon atoms, hydroxy lower alkyl, halo lower alkyl, cycloalkylalkyl of four to about eight carbon atoms, lower alkoxy lower alkyl, phenyl lower alkyl, phenyl, lower alkenyl, lower alkynyl, lower alkenyl lower alkyl, lower alkynyl lower alkyl, carboxy lower alkyl, lower alkanoyl, lower alkoxycarbonyl, carboxy and cyano lower alkyl; wherein each of R⁴ through R⁹ is independently selected from hydrido, lower alkyl, cycloalkyl of three to about eight carbon atoms, cycloalkylalkyl of four to about eight carbon atoms, phenyl lower alkyl, phenyl, lower alkoxy lower alkyl, halo lower alkyl, hydroxy lower alkyl, carboxy, carboxy lower alkyl, lower alkanoyl, lower alkenyl and lower alkynyl; wherein R⁴ and R⁵ may be taken together to form oxo; wherein R⁶ and R⁷ may be taken together to form oxo; wherein R⁸ and R⁹ may be taken together to form oxo; wherein A is selected from phenyl, naphthyl, heteroaryl, phenoxy, naphthyloxy, heteroaryloxy, phenyl lower alkoxy, naphthyl lower alkoxy, heteroaryl lower alkoxy, phenylamino, naphthylamino, heteroarylamino, phenyl lower alkylamino, naphthyl lower alkylamino, heteroaralkylamino, phenylthio, naphthylthio, heteroarylthio, phenyl lower alkylthio and heteroaryl lower alkylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, phenyl lower alkyl, phenyl, lower alkoxy, phenoxy, pheny lower alkoxy, lower alkoxy lower alkyl, halo, halo lower alkyl, hydroxy lower alkyl, cyano, amino, mono lower alkylamino, di lower alkylamino, carboxy, carboxy lower alkyl, lower alkanoyl, lower alkenyl and lower alkynyl; or a pharmaceutically acceptable salt thereof.

A more highly preferred family of such compounds are those compounds wherein each of R¹ and R¹⁰ is selected from hydrido, lower alkyl, cycloalkyl of three to about eight carbon atoms, cycloalkylalkyl of four to about eight carbon atoms, benzyl, phenyl, lower alkenyl lower alkyl and lower alkynyl lower alkyl; wherein each of R² and R³ is independently selected from hydrido, lower alkyl, cycloalkyl of three to about eight carbon atoms, cycloalkylalkyl of four to about eight carbon atoms, benzyl, phenyl, lower alkenyl, lower alkynyl, lower alkenyl lower alkyl, lower alkynyl lower alkyl, lower alkanoyl and lower alkoxycarbonyl; wherein each of R⁴ through R⁹ is independently selected from hydrido, lower alkyl, cycloalkyl of three to about eight carbon atoms, cycloalkylalkyl of four to about eight carbon atoms, benzyl, phenyl, lower alkoxy lower alkyl, halo lower alkyl, hydroxy lower alkyl, lower alkanoyl, lower alkenyl and lower alkynyl; wherein R⁴ and R⁵ may be taken together to form oxo; wherein R⁶ and R⁷ may be taken together to form oxo; wherein R⁸ and R⁹ may be taken together to form oxo; wherein A is selected from phenyl, naphthyl, thienyl, phenoxy, benzyloxy, naphthyloxy, thiophenoxy, phenylamino, benzylamino, naphthylamino, phenylthio, benzylthio and naphthylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, lower alkoxy, lower alkoxy lower alkyl, halo, halo lower alkyl, hydroxy lower alkyl, amino, mono lower alkylamino, di lower alkylamino, lower alkanoyl, lower alkenyl and lower alkynyl; or a pharmaceutically acceptable salt thereof. Compounds of particular interest which fall within the scope of such formula include:
2-[N-[2-(3,4-dichlorophenyl)ethyl]-N-(methyl)aminoethyl]-1-(methyl)piperidine
2-[N-[2-(3,4-dichlorophenyl)ethyl]-N-(methyl)aminoethyl]-1-(methyl) homopiperidine
2-[N-[2-(3,4-dichlorophenyl)ethyl]-N-(methyl)aminoethyl]-1 (methyl)pyrrolidine
2-[N-(3-benzothienyl)ethyl]-N-(methyl)aminoethyl]-1-(methyl)piperidine
2-[N-(3-benzothienyl)ethyl]-N-(methyl)aminoethyl]-1-(methyl)homopiperidine
2-[N-(3-benzothienyl)ethyl]-N-(methyl)aminoethyl]-1-(methyl)pyrrolidine
2-[N-(2-[2-napthyl]ethyl)]-N-(methyl)aminoethyl]-1-(methyl)piperidine
2-[N-(2-[2-napthyl]ethyl)]-N-(methyl)aminoethyl]-1-(methyl)homopiperidine
2-[N-(2-[2-napthyl]ethyl)]-N-(methyl)aminoethyl]-1-(methyl)pyrrolidine
2-[N-(3,4-dichlorophenyl)ethyl]-N-(methyl)aminoethyl]-1-(ethyl)piperidine
2-[N-(3,4-dichlorophenyl)ethyl]-N-(methyl)aminoethyl]-1-(ethyl)homopiperidine
2-[N-(3,4-dichlorophenyl)ethyl]-N-(methyl)aminoethyl]-1-(ethyl)pyrrolidine
2-[N-(3-benzothienyl)ethyl]-N-(methyl)aminoethyl]-1-(ethyl)piperidine
2-[N-(3-benzothienyl)ethyl]-N-(methyl)aminoethyl]-1-(ethyl)homopiperidine
2-[N-(3-benzothienyl)ethyl]-N-(methyl)aminoethyl]-1-(ethyl)pyrrolidine
2-[N-(2-[2-naphthyl]ethyl)]-N-(methyl)aminoethyl]-1-(ethyl)piperidine
2-[N-(2-[2-naphthyl]ethyl)]-N-(methyl)aminoethyl]-1-(ethyl)homopiperidine
2-[N-(2-[2-naphthyl]ethyl)]-N-(methyl)aminoethyl]-1-(ethyl)pyrrolidine
2-[N-(3,4-dichlorophenylethyl)aminomethyl]-1-(ethyl)pyrrolidine
2-[N-(3,4-dichlorophenylethyl)aminomethyl]-1-(ethyl)piperidine
2-[N-(3,4-dichlorophenylethyl)aminomethyl]-1-(ethyl)homopiperidine
2-[N-(3-benxothienyl)ethyl)aminomethyl]-1-(ethyl)pyrrolidine
2-[N-(3-benxothienyl)ethyl)aminomethyl]-1-(ethyl)piperidine
2-[N-(3-benxothienyl)ethyl)aminomethyl]-1-(ethyl)homopiperidine
2-[N-(2-[2-naphthyl]ethyl)aminomethyl]-1-(ethyl)pyrrolidine
2-[N-(2-[2-naphthyl]ethyl)aminomethyl]-1-(ethyl)piperidine
2-[N-(2-[2-naphthyl]ethyl)aminomethyl]-1-(ethyl)homopiperidine

Additional examples of sigma-2 agonists include compounds of the formula: 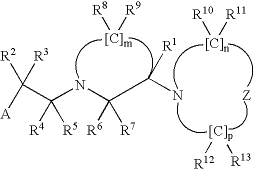
wherein each of R¹, R⁴, R⁵, R⁶ and R⁷ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein each of R², R³ and R⁸ through R¹³ is independently selected from hydrido, hydroxy, alkyl, cycloalkyl, cycloalkylalkyl, aralkyl, aryl, alkoxy, aryloxy, aralkoxy, alkoxyalkyl, haloalkyl, hydroxyalkyl, cyano, amino, monoalkylamino, dialkylamino, carboxy, carboxyalkyl, alkanoyl, alkenyl and alkynyl; wherein R² and R³ may be taken together to form oxo or to form a saturated or partially unsaturated carbocyclic group having three to eight ring carbons; wherein R⁴ and R⁵ may be taken together to form a saturated or partially unsaturated carbocyclic group having three to eight ring carbons; wherein R⁸ and R⁹ may be taken together to form oxo; wherein R¹⁰ and R¹¹ may be taken together to form oxo; wherein each of m, n and p is an integer of from one to four; wherein Z is selected from 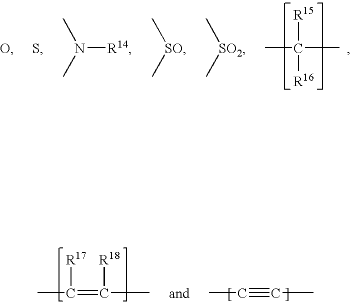
wherein R¹⁴ may be selected from hydrido, alkyl, cycloalkyl, cycloalkylalkyl, aryl, aralkyl, heteroaryl, alkanoyl, aralkanoyl, aroyl, aminoalkyl, monoalkylaminoalkyl and dialkylaminoalkyl; wherein each of R¹⁵ through R¹⁸ is independently selected from hydrido, hydroxy, alkyl, cycloalkyl, cycloalkylalkyl, aralkyl, aryl, alkoxy, aralkoxy, aryloxy, alkoxyalkyl, haloalkyl, hydroxyalkyl, halo, cyano, amino, monoalkylamino, dialkylamino, carboxy, carboxyalkyl and alkanoyl; wherein A is selected from aryl, heteroaryl, aryloxy, heteroaryloxy, aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically-acceptable salt thereof.

A preferred family of such compounds consists of those compounds wherein each of R¹, R⁴, R⁵R⁶ and R⁷ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein each of R², R³ and R⁸ through R¹³ is independently selected from hydrido, hydroxy, alkyl, cycloalkyl, cycloalkylalkyl, aralkyl, aryl, alkoxy, aryloxy, aralkoxy, alkoxyalkyl, haloalkyl, hydroxyalkyl, cyano, amino, monoalkylamino, dialkylamino, carboxy, carboxyalkyl, alkanoyl, alkenyl and alkynyl; wherein R² and R³ may be taken together to form oxo or to form a saturated or partially unsaturated carbocyclic group having three to eight ring carbons; wherein R⁴ and R⁵ may be taken together to form oxo or to form a saturated or partially unsaturated carbocyclic group having three to eight ring carbons; wherein each in, n and p is an integer of from one to four; wherein Z is selected from 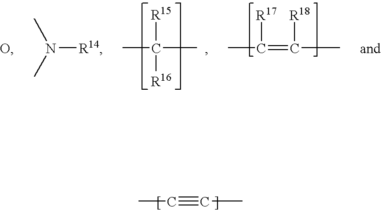
wherein R¹⁴ may be selected from hydrido, alkyl, cycloalkyl, cycloalkylalkyl, aryl, aralkyl, heteroaryl, alkanoyl, aralkanoyl and aroyl; wherein each of R¹⁵ through R¹⁸ is independently selected from hydrido, alkyl, cycloalkyl, cycloalkylalkyl, aralkyl, aryl, alkoxyalkyl, haloalkyl, hydroxyalkyl, halo, cyano, carboxy, carboxyalkyl and alkanoyl; wherein A is selected from aryl, heteroaryl, aryloxy, heteroaryloxy, aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically acceptable salt thereof.

A more preferred family of such compounds consists of those compounds wherein each of R¹, R⁴, R⁵, R⁶ and R⁷ is independently selected from hydrido, lower alkyl, cycloalkyl of three to about eight carbon atoms, phenyl lower alkyl, phenyl, lower alkoxy lower alkyl, halo lower alkyl; hydroxy lower alkyl, carboxy, carboxy lower alkyl, lower alkanyl, lower alkenyl, lower alkynyl; wherein R², R³ and R⁸ through R¹³ is independently selected from hydrido, hydroxy, lower alkyl, cycloalkyl of three to about eight carbon atoms, cycloalkylalkyl of four to about eight carbon atoms, phenyl lower alkyl, phenyl, lower alkoxy, phenoxy, phenyl lower alkoxy, lower alkoxy lower alkyl, halo lower alkyl, hydroxy lower alkyl, cyano, amino, mono lower alkylamino, di lower alkylamino, carboxy, carboxy lower alkyl, lower alkanoyl, lower alkenyl and lower alkynyl; wherein R² and R³ may be taken together to form a saturated or partially unsaturated carbocyclic group having three to eight ring carbons; wherein R⁴ and R⁵ may be taken together to form a saturated or partially unsaturated carbocyclic group having three to eight ring carbons; wherein each of n and m is a number selected from one through four; wherein each of m, n and p is an integer from one to four; wherein Z is selected from 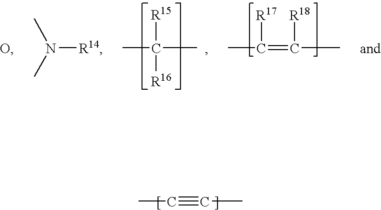
wherein R¹⁴ may be selected from hydrido, lower alkyl, cycloalkyl of three to about eight carbon atoms, cycloalkylalkyl of four to about eight carbon atoms, phenyl, phenyl lower alkyl, heteroaryl, lower alkanoyl, phenylalkanoyl, benzoyl, amino lower alkyl, mono lower alkyl-amino lower alkyl and di lower alkylamino-lower alkyl; wherein each of R¹⁵ through R¹⁸ is independently selected from hydrido, hydroxy, lower alkyl, cycloalkyl of three to about eight carbon atoms, cycloalkylalkyl of four to about eight carbon atoms, pheny lower alkyl, phenyl, lower alkoxy lower alkyl, halo lower alkyl, hydroxy lower alkyl, halo, cyano, amino, mono lower alkylamino, di lower alkylamino, carboxy, carboxy lower alkyl and lower alkanoyl; wherein A is selected from phenyl, naphthyl, heteroaryl, phenoxy, naphthyloxy, heteroaryloxy, phenyl lower alkoxy, naphthyl lower alkoxy, heteroaryl lower alkoxy, phenylamino, naphthylamino, heteroarylamino, phenyl lower alkylamino, naphthyl lower alkylamino, heteroaralkylamino, phenylthio, naphthylthio, heteroarylthio, phenyl lower alkylthio and heteroaryl lower alkylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, phenyl lower alkyl, phenyl, lower alkoxy, phenoxy, pheny lower alkoxy, lower alkoxy lower alkyl, halo, halo lower alkyl, hydroxy lower alkyl, cyano, amino, mono lower alkylamino, di lower alkylamino, carboxy, carboxy lower alkyl, lower alkanoyl, lower alkenyl and lower alkynyl; or a pharmaceutically acceptable salt thereof.

A more highly preferred family of such compounds consists of those compounds wherein each of R¹, R⁴, R⁵, R⁶ and R⁷ is independently selected from hydrido, lower alkyl, cycloalkyl of three to about eight carbon atoms, cycloalkylalkyl of four to about eight carbon atoms, benzyl, phenyl, lower alkoxy lower alkyl, halo lower alkyl, hydroxy lower alkyl, lower alkanoyl, lower alkenyl, and lower alkynyl; wherein R², R³ and R⁸ through R¹³ is independently selected from hydrido, hydroxy, lower alkyl, cycloalkyl of three to about eight carbon atoms, cycloalkylalkyl of four to about eight carbon atoms, benzyl, phenyl, lower alkoxy, phenoxy, benzyloxy, lower alkoxy lower alkyl, halo lower alkyl, hydroxy lower alkyl, lower alkanoyl, lower alkenyl and lower alkynyl; wherein R² and R³ may be taken together to form a saturated or partially unsaturated carbocyclic group having three to eight ring carbons; wherein R⁴ and R⁵ may be taken together to form oxo or to form a saturated or partially unsaturated carbocyclic group having three to eight ring carbons; wherein each of m, n and p is an integer from one to four; wherein Z is selected from 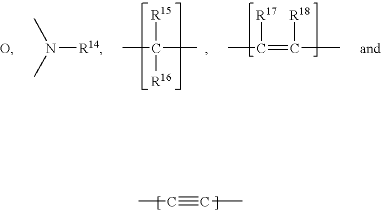
wherein R¹⁴ may be selected from hydrido, lower alkyl, cycloalkyl of three to about eight carbon atoms, cycloalkylalkyl of four to about eight carbon atoms, phenyl and benzyl; wherein each of R¹⁵ through R¹⁸ is independently selected from hydrido, lower alkyl, cycloalkyl of three to about eight carbon atoms, cycloalkylalkyl of four to about eight carbon atoms, benzyl, phenyl, lower alkoxy lower alkyl, hydroxy lower alkyl and halo; wherein A is selected from phenyl, naphthyl, benzo[b]thienyl, thienyl, phenoxy, benzyloxy, naphthyloxy, thiophenoxy, phenylamino, benzylamino, naphthylamino, phenylthio, benzylthio and naphthylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, lower alkoxy, lower alkoxy lower alkyl, halo, halo lower alkyl, hydroxy lower alkyl, amino, mono lower alkylamino, di lower alkylamino, lower alkanoyl, lower alkenyl and lower alkynyl; or a pharmaceutically acceptable salt thereof.

A family of compounds of particular interest are compounds embraced by the formula: 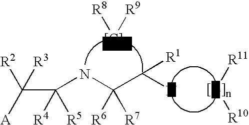
wherein each of R¹, R⁴, R⁵, R⁶ and R⁷ is independently selected from hydrido, lower alkyl, benzyl and halo lower alkyl; wherein R², R³ and R⁸ through R¹¹ is independently selected from hydrido, hydroxy, lower alkyl, benzyl, phenoxy, benzyloxy and halo lower alkyl; wherein n is an integer of from four to six; wherein m is an integer of from two to four; wherein A is selected from phenyl, naphthyl, benzothienyl, benzofuranyl and thienyl; wherein any of the foregoing A groups can be further substituted with one or more substituents independently selected from hydrido, hydroxy, lower alkyl, lower alkoxy, halo, halo lower alkyl, amino, mono lower alkylamino and di lower alkylamino; or a pharmaceutically acceptable salt thereof.

A more preferred family of such compounds consists of compounds wherein each of R¹, R⁴, R⁵, R⁶ and R⁷ is independently selected from hydrido, methyl, ethyl, propyl, benzyl, and halo lower alkyl, wherein R², R³ and R⁸ through R¹³ is independently selected from hydrido, hydroxy, methyl, ethyl, propyl, benzyl, phenoxy, benzyloxy and halo lower alkyl; wherein m is a number selected from four or five; wherein m is an integer of from two or three; wherein A is phenyl or naphthyl; wherein any of the foregoing A groups can be further substituted with one or more substituents independently selected from hydroxy, methyl, ethyl, propyl, methoxy, ethoxy, methylenedioxy, halo, trifluoromethyl, amino, methylamino and dimethylamino; or a pharmaceutically acceptable salt thereof. Of particular interest are the following specific compounds:
3-(1-pyrrolidinyl)-N-(2-[3,4-dichlorophenyl]ethyl)piperidine
3-(1-piperidinyl)-N-(2-[3,4-dichlorophenyl]ethyl)piperidine
3-(1-pyrrolidinyl)-N-(2-[3,4-dichlorophenyl]ethyl)homopiperidine
3-(1-piperidinyl)-N-(2-[3,4-dichlorophenyl]ethyl)homopiperidine
3-(1-pyrrolidinyl)-N-(2-[3,4-dichlorophenyl]ethyl)pyrrolidine
3-(1-piperidinyl)-N-(2-[3,4-dichlorophenyl]ethyl)pyrrolidine
3-(1-homopiperidinyl)-N-(2-[3,4-dichlorophenyl]ethyl)piperidine
3-(1-homopiperidinyl)-N-(2-[3,4-dichlorophenyl]ethyl)homopiperidine
3-(1-homopiperidinyl)-N-(2-[3,4-dichlorophenyl]ethyl)pyrrol idine
3-(1-homopiperidinyl)-N-(2-[3-benzothienyl]ethyl)piperidine
3-(1-homopiperidinyl)-N-(2-[3-benzothienyl]ethyl)homopiperidine
3-(1-homopiperidinyl)-N-(2-[3-benzothienyl]ethyl)pyrrolidine
3-(1-homopiperidinyl)-N-(2-[2-naphthyl]ethyl)piperidine
3-(1-homopiperidinyl)-N-(2-[1-naphthyl]ethyl)homopiperidine
3-(1-homopiperidinyl)-N-(2-[3-naphthyl]ethyl)pyrrolidine
3-(1-piperidinyl)-N-(2-[3-benzothienyl]ethyl)piperidine
3-(1-pyrrolidinyl)-N-(2-[3-benzothienyl]ethyl)piperidine
3-(1-piperidinyl)-N-(2-[3-benzothienyl]ethyl)pyrrolidine
3-(1-pyrrolidinyl)-N-(2-[3-benzothienyl]ethyl)pyrrolidine
3-(1-piperidinyl)-N-(2-[3-benzothienyl]ethyl)homopiperidine
3-(1-pyrrolidinyl)-N-(2-[3-benzothienyl]ethyl)homopiperidine

Additional examples of sigma-2 agonists include compounds having the formula: 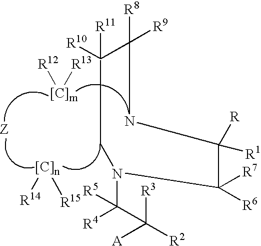
wherein each of R, R¹, R⁴, R⁵, R⁶ and R⁷ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein each of R², R³ and R¹⁰ through R¹⁵ is independently selected from hydrido, hydroxy, alkyl, cycloalkyl, cycloalkylalkyl, aralkyl, aryl, alkoxy, aryloxy, aralkoxy, alkoxyalkyl, haloalkyl, hydroxyalkyl, cyano, amino, monoalkylamino, dialkylamino, carboxy, carboxyalkyl, alkanoyl, alkenyl and alkynyl; wherein each of R⁸ and R⁹ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein R and R¹ may be taken together to form oxo or to form a saturated or partially unsaturated carbocyclic group having three to eight ring carbons; wherein R² and R³ may be taken together to form oxo or to form a saturated or partially unsaturated carbocyclic group having three to eight ring carbons; wherein R⁴ and R⁵ may be taken together to form oxo or to form a saturated or partially unsaturated carbocyclic group having three to eight ring carbons; wherein R¹² and R¹³ may be taken together to form oxo; wherein R¹⁴ and R¹⁵ may be taken together to form oxo; wherein each of m and n is an integer of from one to four; wherein Z is selected from 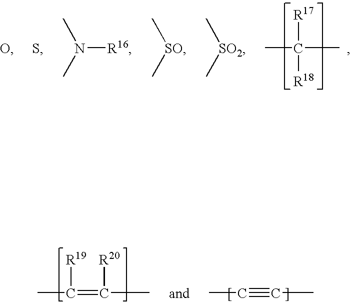
wherein R¹⁶ is selected from hydrido, alkyl, cycloalkyl, cycloalkylalkyl, aryl, aralkyl, heteroaryl, alkanoyl, aralkanoyl, aroyl, aminoalkyl, monoalkylaminoalkyl and dialkylaminoalkyl; wherein each of R¹⁷ and R¹⁸ is independently selected from hydrido, hydroxy, alkyl, cycloalkyl, cycloalkylalkyl, aralkyl, aryl, alkoxy, aralkoxy, aryloxy, alkoxyalkyl, haloalkyl, hydroxyalkyl, halo, cyano, amino, monoalkylamino, dialkylamino, carboxy, carboxyalkyl and alkanoyl; wherein each of R¹⁹ and R²⁰ is independently selected from hydrido, alkyl, cycloalkyl, cycloalkylalkyl, aralkyl, alkoxyalkyl, haloalkyl, hydroxyalkyl, halo, cyano, carboxy, carboxyalkyl and alkanoyl; wherein A is selected from aryl, heteroaryl, aryloxy, heteroaryloxy, aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically-acceptable salt thereof.

A preferred family of such compounds consists of those compounds wherein each of R, R¹, R⁴, R⁵, R⁶ and R⁷ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein each of R², R³ and R¹⁰ through R¹⁵ is independently selected from hydrido, hydroxy, alkyl, cycloalkyl, cycloalkylalkyl, aralkyl, aryl, alkoxy, aryloxy, aralkoxy, alkoxyalkyl, haloalkyl, hydroxyalkyl, cyano, amino, monoalkylamino, dialkylamino, carboxy, carboxyalkyl, alkanoyl, alkenyl and alkynyl; wherein each of R⁸ and R⁹ is independently selected from hydrido, alkyl, cycloalkyl, cycloalkylalkyl, aralkyl, aryl, alkoxyalkyl, haloalkyl, hydroxyalkyl, carboxy, carboxyalkyl, alkanoyl, alkenyl and alkynyl; wherein R and R¹ my be taken together to form oxo or to form a saturated or partially unsaturated carbocyclic group having three to eight ring carbons; wherein R² and R³ may be taken together to form oxo or to form a saturated or partially unsaturated carbocyclic group having three to eight ring carbons; wherein R⁴ and R⁵ may be taken together to form oxo or to form a saturated or partially unsaturated carbocyclic group having three to eight ring carbons; wherein R¹² and R¹³ may be taken together to form oxo; wherein R¹⁴ and R¹⁵ may be taken together to form oxo; wherein each of m and n is an integer from one to four; wherein Z is selected from 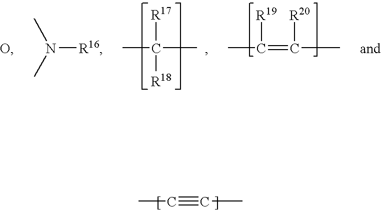
wherein R¹⁶ may be selected from hydrido, alkyl, cycloalkyl, cycloalkylalkyl, aryl, aralkyl, heteroaryl, alkanoyl, aralkanoyl and aroyl; wherein each of R¹⁷ through R²⁰ is independently selected from hydrido, alkyl, cycloalkyl, cycloalkylalkyl, aralkyl, aryl, alkoxyalkyl, haloalkyl, hydroxyalkyl, carboxyalkyl and alkanoyl; wherein A is selected from aryl, heteroaryl, aryloxy, heteroaryloxy, aralkoxy, heteroaralkoxy, arylamino, heteroarylamino, aralkylamino, heteroaralkylamino, arylthio, heteroarylthio, aralkylthio and heteroaralkylthio; wherein any of the foregoing A groups can be further substituted with one or more substituents independently selected from hydrido, hydroxy, alkyl, cycloalkyl, cycloalkylalkyl, aralkyl, aryl, alkoxy, aryloxy, aralkoxy, alkoxyalkyl, halo, haloalkyl, hydroxyalkyl, cyano, amino, monoalkylamino, dialkylamino, carboxy, carboxyalkyl, alkanoyl, alkenyl and alkynyl; or a pharmaceutically acceptable salt thereof.

A more preferred family of such compounds consists of those compounds wherein each of R, R¹, R⁴, R⁵, R⁶ and R⁷ is independently selected from hydrido, lower alkyl, cycloalkyl of three to about eight carbon atoms, phenyl lower alkyl, phenyl, lower alkoxy lower alkyl, halo lower alkyl; hydroxy lower alkyl, carboxy, carboxy lower alkyl, lower alkanyl, lower alkenyl, lower alkynyl; wherein R², R³ and R¹⁰ through R¹⁵ is independently selected from hydrido, hydroxy, lower alkyl, cycloalkyl of three to about eight carbon atoms, cycloalkylalkyl of four to about eight carbon atoms, phenyl lower alkyl, phenyl, lower alkoxy, phenoxy, phenyl lower alkoxy, lower alkoxy lower alkyl, halo lower alkyl, hydroxy lower alkyl, cyano, amino, mono lower alkylamino, di lower alkylamino, carboxy, carboxy lower alkyl, lower alkanoyl, lower alkenyl and lower alkynyl; wherein each of R⁸ and R⁹ is independently selected from hydrido, lower alkyl, cycloalkyl of three to about eight carbon atoms, phenyl lower alkyl, phenyl, lower alkoxy lower alkyl, halo lower alkyl, hydroxy lower alkyl, carboxy, carboxy lower alkyl, lower alkanoyl, lower alkenyl and lower alkynyl; wherein R² and R³ may be taken together to form a saturated or partially unsaturated carbocyclic group having three to eight ring carbons; wherein R⁴ and R⁵ may be taken together to form a saturated or partially unsaturated carbocyclic group having three to eight ring carbons; wherein R¹² and R¹³ may be taken together to form oxo; wherein R¹⁴ and R¹⁵ may be taken together to form oxo; wherein m is an integer from 2-4 and n and p are integers of from one to four; wherein Z is selected from 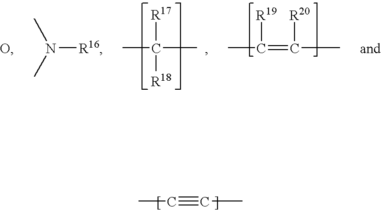
wherein R¹⁶ may be selected from hydrido, lower alkyl, cycloalkyl of three to about eight carbon atoms, cycloalkylalkyl of four to about eight carbon atoms, phenyl, phenyl lower alkyl, heteroaryl, lower alkanoyl, phenylalkanoyl, benzoyl, amino lower alkyl, mono lower alkyl-amino lower alkyl and di lower alkylamino-lower alkyl; wherein each of R¹⁷ and R¹⁸ is independently selected from hydrido, hydroxy, lower alkyl, cycloalkyl of three to about eight carbon atoms, cycloalkylalkyl of four to about eight carbon atoms, pheny lower alkyl, phenyl, lower alkoxy lower alkyl, halo lower alkyl, hydroxy lower alkyl, halo, cyano, amino, mono lower alkylamino, di lower alkylamino, carboxy, carboxy lower alkyl and lower alkanoyl; wherein each of R¹⁹ and R²⁰ is independently selected from hydrido, lower alkyl, cycloalkyl of three to about eight carbon atoms, cycloalkylalkyl of four to about eight carbon atoms, phenyl lower alkyl, phenyl, lower alkoxy lower alkyl, halo lower alkyl, hydroxy lower alkyl, halo, cyano, carboxy, carboxy lower alkyl and lower alkanoyl; wherein A is selected from phenyl, naphthyl, heteroaryl, phenoxy, naphthyloxy, heteroaryloxy, phenyl lower alkoxy, naphthyl lower alkoxy, heteroaryl lower alkoxy, phenylamino, naphthylamino, heteroarylamino, phenyl lower alkylamino, naphthyl lower alkylamino, heteroaralkylamino, phenylthio, naphthylthio, heteroarylthio, phenyl lower alkylthio and heteroaryl lower alkylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, phenyl lower alkyl, phenyl, lower alkoxy, phenoxy, pheny lower alkoxy, lower alkoxy lower alkyl, halo, halo lower alkyl, hydroxy lower alkyl, cyano, amino, mono lower alkylamino, di lower alkylamino, carboxy, carboxy lower alkyl, lower alkanoyl, lower alkenyl and lower alkynyl; or a pharmaceutically acceptable salt thereof.

A more highly preferred family of such compounds consists of those-compounds wherein each of R, R¹, R⁴, R⁵, R⁶ and R⁷ is independently selected from hydrido, lower alkyl, cycloalkyl of three to about eight carbon atoms, cycloalkylalkyl of four to about eight carbon atoms, benzyl, phenyl, lower alkoxy lower alkyl, halo lower alkyl, hydroxy lower alkyl, lower alkanoyl, lower alkenyl, and lower alkynyl; wherein R², R³ and R¹⁰ through R¹⁵ is independently selected from hydrido, hydroxy, lower alkyl, cycloalkyl of three to about eight carbon atoms, cycloalkylalkyl of four to about eight carbon atoms, benzyl, phenyl, lower alkoxy, phenoxy, benzyloxy, lower alkoxy lower alkyl, halo lower alkyl, hydroxy lower alkyl, lower alkanoyl, lower alkenyl and lower alkynyl; wherein each of R⁸ and R⁹ is independently selected from hydrido, lower alkyl, cycloalkyl of three to about eight carbon atoms, phenyl lower alkyl, phenyl, lower alkoxy lower alkyl, halo lower alkyl, hydroxy lower alkyl, carboxy, carboxy lower alkyl, lower alkanoyl, lower alkenyl and lower alkynyl; wherein R² and R³ may be taken together to form a saturated or partially unsaturated carbocyclic group having three to eight ring carbons; wherein R⁴ and R⁵ may be taken together to form a saturated or partially unsaturated carbocyclic group having three to eight ring carbons; wherein R¹² and R¹³ may be taken together to form oxo; wherein each of m and n is an integer of from one to four; wherein Z is selected from 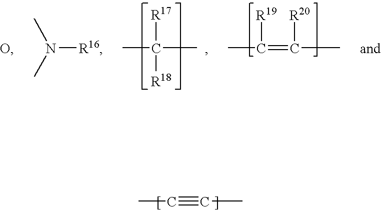
wherein R¹⁶ may be selected from hydrido, lower alkyl, cycloalkyl of three to about eight carbon atoms, cycloalkylalkyl of four to about eight carbon atoms, phenyl and benzyl; wherein each of R¹⁷ through R²⁰ is independently selected from hydrido, lower alkyl, cycloalkyl of three to about eight carbon atoms, cycloalkylalkyl of four to about eight carbon atoms, benzyl, phenyl, lower alkoxy lower alkyl, hydroxy lower alkyl and halo; wherein A is selected from phenyl, naphthyl, benzo[b]thienyl, thienyl, phenoxy, benzyloxy, naphthyloxy, thiophenoxy, phenylamino, benzylamino, naphthylamino, phenylthio, benzylthio and naphthylthio; wherein any of the foregoing A groups can be further substituted with one or more substituents independently selected from hydrido, hydroxy, lower alkyl, cycloalkyl of three to about eight carbon atoms, cycloalkylalkyl of four to about eight carbon atoms, lower alkoxy, lower alkoxy lower alkyl, halo, halo lower alkyl, hydroxy lower alkyl, amino, mono lower alkylamino, di lower alkylamino, lower alkanoyl, lower alkenyl and lower alkynyl; or a pharmaceutically acceptable salt thereof.

A family of such compounds of particular interest are compounds embraced by the formula: 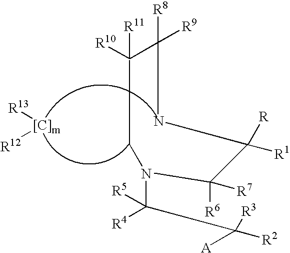
wherein each of R, R¹, R⁴, R⁵, R⁶ and R⁷ is independently selected from hydrido, lower alkyl, benzyl and halo lower alkyl; wherein each of R², R³ and R¹⁰ through R¹³ is independently selected from hydrido, hydroxy, lower alkyl, benzyl, phenoxy, benzyloxy and halo lower alkyl; wherein each of R⁸ and R⁹ is independently selected from hydrido, lower alkyl, benzyl and halo lower alkyl; wherein m is an integer of from two to four; wherein A is selected from phenyl, naphthyl, benzothienyl, benzofuranyl and thienyl; wherein any of the foregoing A groups can be further substituted with one or more substituents independently selected from hydrido, hydroxy, lower alkyl, lower alkoxy, halo, halo lower alkyl, amino, mono lower alkylamino and di lower alkylamino; or a pharmaceutically acceptable salt thereof.

A more preferred family of such compounds consists of compounds wherein each of R, R¹, R⁴, R⁵, R⁶ and R⁷ is independently selected from hydrido, methyl, ethyl, propyl, benzyl, and halo lower alkyl, wherein each of R², R³ and R¹⁰ through R¹³ is independently selected from hydrido, hydroxy, methyl, ethyl, propyl, benzyl, phenoxy, benzyloxy and halo lower alkyl; wherein each of R⁸ and R⁹ is independently selected from hydrido, methyl, ethyl, propyl, benzyl and halo lower alkyl; wherein m is an integer of from two or three; wherein A is phenyl or naphthyl; wherein any of the foregoing A groups can be further substituted with one or more substituents independently selected from hydroxy, methyl, ethyl, propyl, methoxy, ethoxy, methylenedioxy, halo, trifluoromethyl, amino, methylamino and dimethylamino; or a pharmaceutically acceptable salt thereof. Of particular interest are the following specific compounds:
4-[2-(3,4-dichlorophenyl)ethyl]1,4-diazabicyclo[2.2.3]nonane;
4-[2-(3-benzothienyl)ethyl]-1,4-diazabicyclo[2.2.3]nonane;
4-[2-naphthylethyl]-1,4-diazabicyclo[2.2.3]nonane;
4-[2-(3,4-dichlorophenyl)ethyl-1,4-diazabicyclo[3.2.3]decane;
4-[2-(3-benzothienyl)ethyl]-1,4-diazabicyclo[4.2.3]undecane;
4-[2-naphthylethyl]-1,4-diazabicyclo[5.2.3]dodecane;

Additional examples of sigma-2 agonists include 5-substituted morphan-7-ones, 5,8-disubstituted morphan-7-ones, iboga alkaloids such as 12-methoxyibogamine (ibogaine) and 13-methoxyibogamine and the like (Bowen, W. D. et al. (1995) “IBOGAINE AND ITS CONGENERS ARE SIGMA-2 RECEPTOR-SELECTIVE LIGANDS WITH MODERATE AFFINITY,” Eur. J. Parmacol. 279:RI-R3, Bowen, W. D. (2001) “SIGMA RECEPTORS AND IBOGA ALKALOIDS,” Alkaloids Chem. Biol. 56:173-91), pentazocine (racemic pentzocine, Talwin) and haloperidol. Preferred sigma-2 selective ligands include, but are not limited to, (+)-5,8-disubstituted morphan-7-ones and iboga alkaloids. Preferred (+)-5,8-disubstituted morphan-7-ones are CB-64D and CB-184. CB-184 is a particularly preferred sigma-2 agonist. CB-184 is (+)-1R,5R^{-E-}8-(3,4-dichlorobenzylidene)-5-(3-hydroxyphenyl)-2-methylmorphan-7-one.

The 5,8-disubstituted morphan-7-ones are illustrated by the following general formula: 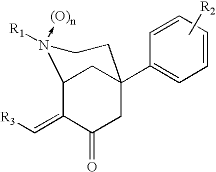
wherein:
n=0 or 1;
R₁=lower-alkyl, lower-alkenyl, cycloalkyl, lower-alkynyl, lower-alkylaryl, or hydrogen;
R₂=lower-alkyl, lower-alkoxy, lower-alkylamino, hydroxy, amino, nitro, halo, azido, or hydrogen
R₃=aryl, alkylaryl, lower-alkyl, cycloalkyl, lower-alkenyl, lower-alkynyl, lower-alkylaryl, lower-alkoxy, lower-alkylamino, hydrogen, or hydroxy.

The term “hydrido” denotes a single hydrogen atom (H). This hydrido group may be attached, for example, to an oxygen atom to form a hydroxyl group; or as another example, two hydrido groups may be attached to a carbon atom to form a divalent —CH₂— group, that is, a “methylene” group; or as another example, one hydrido group may be attached to a carbon atom to form a trivalent —CH< group. Where the term “alkyl” is used, either alone or within other terms such as “haloalkyl” and “hydroxyalkyl”, the term “alkyl” embraces linear or branched radicals having one to about twenty carbon atoms or, preferably, one to about ten carbon atoms. More preferred alkyl radicals are “lower alkyl” radicals having one to about five carbon atoms. The term “cycloalkyl” embraces cyclic radicals having three to about six carbon atoms, such as cyclopropyl and cyclobutyl. The term “haloalkyl” embraces radicals wherein any one or more of the alkyl carbon atoms is substituted with one or more halo groups, preferable selected from bromo, chloro and fluoro. Specifically embraced by the term “haloalkyl” are monohaloalkyl, dihaloalkyl and polyhaloalkyl groups. A monohaloalkyl group, for example, may have either a bromo, a chloro, or a fluoro atom within the group. Dihaloalkyl and polyhaloalkyl groups may be substituted with two or more of the same halo groups, or may have a combination of different halo groups. A dihaloalkyl group, for example, may have two bromo atoms, such as a dibromomethyl group, or two chloro atoms, such as a dichloromethyl group, or one bromo atom and one chloro atom, such as a bromochloromethyl group. An example of a polyhaloalkyl is a trifluoromethyl group. The terms “alkylol” and “hydroxyalkyl” embrace linear or branched alkyl groups having one to about ten carbon atoms any one of which may be substituted with one or more hydroxyl groups. The term “alkenyl” embraces linear or branched radicals having two to about twenty carbon atoms, preferable two to about ten carbon atoms, and containing at least one carbon-carbon triple bond. The terms “cycloalkenyl” and “cycloalkynyl” embrace cyclic radicals having three to about ten ring carbon atoms including, respectively, one or more double or triple bonds involving adjacent ring carbons. The terms “alkoxy” and “alkoxyalkyl” embrace linear or branched oxy-containing radicals each having alkyl portions of one to about ten carbon atoms, such as methoxy group. The “alkoxy” or “alkoxyalkyl” radicals may be further substituted with one or more halo atoms, such as fluoro, chloro or bromo, to provide haloalkoxy or haloalkoxyalkyl groups. The term “heteroaryl” embraces aromatic ring systems containing one or two hetero atoms selected from oxygen, nitrogen and sulfur in a ring system having five or six ring members, examples of which are thienyl, furanyl, pyridinyl, thiazolyl, pyrimidyl and isoxazolyl. The term “alkylene chain” describes a chain of two to six methylene (—CH₂—) groups which may form a cyclic structure with or without a hetero atom in the cyclic structure.

Specific examples of alkyl groups are methyl, ethyl, n-propyl, isopropyl, n-butyl, sec-butyl, iso-butyl, tert-butyl, n-pentyl, iso-pentyl, methyl-butyl, dimethylbutyl and neopentyl. Typical alkenyl and alkynyl groups may have one unsaturated bond, such as an allyl group, or may have a plurality of unsaturated bonds, with such plurality of bonds either adjacent, such as allene-type structures, or in conjugation, or separated by several saturated carbons.

The tem “lower-alkyl” as used herein means linear or branched hydrocarbon chains having from one to about ten carbon atoms and thus includes methyl, ethyl, propyl, isopropyl, n-butyl, see-butyl, and the like.

The term “lower-alkenyl” means branched or unbranched unsaturated hydrocarbon radicals of from two to about ten carbon atoms and thus includes 1-ethenyl, 1-propenyl, 2-propenyl, 1-methyl-2-propenyl, isopropenyl, 2-butenyl, isobutenyl, and the like.

The term “cycloalkyl” means bridged or unbridged hydrocarbon ring systems having from three to about ten carbon atoms and thus includes cyclopropyl, cyclobutyl, cyclopentyl, cyclohexyl, cycloheptyl, cyclooctyl, cyclodecyl, norbornyl, adamantyl, and the like.

The term “lower-alkynyl” as used herein means branched or unbranched unsaturated hydrocarbon radicals of from two to about ten carbon atoms and thus includes 1-ethynyl, 1-propynyl, 2-propynyl, 2-butynyl, 1-methyl-2-propynyl, and the like.

The term “alkaryl” as used herein means a substituted or non-substituted aryl wherein the substitution can be hydrogen or one to about 10 carbon atoms.

The term “lower-alkoxy” as used herein means linear or branched alkyloxy substituents having from one to about ten carbon atoms and thus includes methoxy, ethoxy, propoxy, isopropoxy, butoxy, sec-butoxy, and the like.

The term “lower-alkylamino” as used herein means linear or branched alkylamino substituents having from one to about ten carbon atoms and thus includes methylamino, ethylamino, propylamino, isopropylamino, butylamino, sec-butylamino, and the like.

The terms “halogen,” “halide,” or “halo” as used herein mean bromine, chlorine, iodine or fluorine.

The term “aryl” as used herein means optionally substituted phenyl, optionally substituted naphthyl, optionally substituted thienyl, optionally substituted furyl, optionally substituted imidizoyl, optionally substituted pyridyl, optionally substituted thienyl, indolyl, optionally substituted quinolyl, and the like.

The sigma-2 agonists of the invention may also be those analogues of phenylalkylpiperidine compounds (or analogues of the salts thereof) that decrease cellular production of sphingomyelin when provided to recipient cells, and inhibit HIV replication. Phenylalkylpiperidine compounds have the structure: 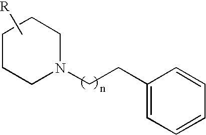
where n is 1 or 2, and R is hydrogen, 3-methyl or 4-methyl; or 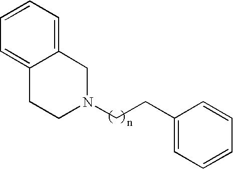
where n is 1 or 2; or 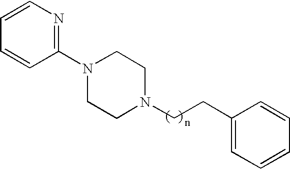
where n is 1 or 2.

Phenylpiperidine ligands of the sigma-2 receptor have been described in the scientific literature (Maeda, D. Y. et al. (2002) “N-ARYLALKYLPIPERIDINES AS HIGH-AFFINITY SIGMA-1 AND SIGMA-2 RECEPTOR LIGANDS: PHENYLPROPYLAMINES AS POTENTIAL LEADS FOR SELECTIVE SIGMA-2 AGENTS,” Bioorg. Med. Chem. Lett. February 11; 12(3):497-500). Some of these compounds have shown agonist activity, while others in this class have shown antagonist activity or may be partial agonists. Agonists can be distinguished from antagonists by those skilled in the art.

It will be appreciated that the sigma-2 agonists of the present invention can possess one or more asymmetric carbon atoms and are thus capable of existing in a number of stereoisomeric forms, i.e. enantiomers and diastereomers. Unless otherwise specified herein, the invention is intended to extend to each of these stereoisomeric forms and to mixtures thereof, including the racemates. In some cases there may be advantages, i.e. greater potency, to using a particular enantiomer when compared to the other enantiomer or the racemate in the methods of the instant invention and such advantages can be readily determined by those skilled in the art. The separate enantiomers may be synthesized from chiral starting materials or the racemates maybe resolved by conventional procedures which are well known in the art of chemistry such as chiral chromatography, fractional crystallization of diastereomeric salts and the like. Likewise, the diastereomers can be separated by conventional procedures which are well known in the art of chemistry such as chromatography, fractional crystallization and the like.

The synthesis of the morphanones is known to those of skill in the art. For example, several groups (Bertha, C. M. et al. (1994) “A MARKED CHANGE OF RECEPTOR AFFINITY OF THE 2-METHYL (3-HYDROXYPHENYL) MORPHANS UPON ATTACHMENT OF AN (E) BENZYLIDENE MOIETY: SYNTHESIS AND EVALUATION OF A NEW CLASS OF EL RECEPTOR LIGANDS,” J. Med. Chem. 37:3163-70; Bertha, C. M. et al. (1995) “(E)-8-BENZYLIDENE DERIVATIVES OF 2-METHYL (3-HYDROXYPHENYL) MORPHANS: HIGHLY SELECTIVE LIGANDS FOR THE σ₂ RECEPTOR SUBTYPE,” J. Med. Chem. 38:4776-85; Bowen, W. D. et al. (1995) “CB-64D and CB-184: LIGANDS WITH HIGH SIGMA-2 RECEPTOR AFFINITY AND SUBTYPE SELECTIVITY,” Eur. J. Parmacol. 278:257-260) describe the preparation of a number of morphanones within this class of compounds as well as the structure activity relationships of these compounds. Dextro-isomers of the morphanones have a high degree of selectivity for the sigma-2 receptor subtype.

When designing and synthesizing a sigma-2 agonist for use according to the present invention, a consideration of the lipophilicity and basicity of the sigma-2 agonist should be made, most preferably so as to permit the sigma-2 agonist to cross the cell membrane and interact with sigma-2 receptors located in sub-cellular organelles (Bowen, W. D. et al. (1999) “SIGMA-2 RECEPTORS THAT MODULATE CALCIUM AND INDUCE CYTOTOXICITY ARE LOCALIZED INTRACELLULARLY,” Soc. Neurosci. Abstr. 25:1708, #680.19). Therefore, sigma-2 agonists that are more lipophilic and have fewer sites available for protonation are preferred for use according to this invention.

The present invention additionally provides a method of treating immunodeficiency virus infection which comprises administering an effective amount of a sigma-2 agonist, and more preferably a sigma-2 selective agonist to a mammal either infected with such virus or at risk of such infection. Thus, the invention provides a treatment for: humans either infected with HIV or at risk of such infection; felines either infected with feline immunodeficiency virus or at risk of such infection; and simians either infected with simian immunodeficiency virus or at risk of such infection.

The effective amount of the sigma-2 agonist to be administered to the mammal, such as a human, is a subtoxic amount. Preferably, the subtoxic amount is an amount that produces little or no killing of uninfected cells. More preferably, the subtoxic amount is an amount that produces little or no effect on the morphology of uninfected cells. The sigma-2 agonist can be administered in a single dose or in multiple doses in a given period of time (e.g., a single daily dose or two or more doses a day). The subtoxic dose depends on the age, weight, general health, and extent of infection being treated. The sigma-2 agonists of the present invention may be administered alone, or in combination with other immunodeficiency virus treatment regimens.

A composition is said to be “pharmacologically acceptable” if its administration can be tolerated by a recipient patient. An agent is physiologically significant if its presence results in a detectable change in the physiology of a recipient patient. The administration of such compounds may be for either a “prophylactic” or “therapeutic” purpose. The compositions of the present invention are said to be administered in a “therapeutically effective amount” if the amount administered is physiologically significant to provide a therapy for an actual infection. When provided therapeutically, the compound is preferably provided at (or shortly after) the onset of a symptom of actual infection. The therapeutic administration of the compound serves to attenuate any actual infection. The compositions of the present invention are said to be administered in a “prophylactically effective amount” if the amount administered is physiologically significant to provide a therapy for an potential infection. When provided prophylactically, the compound is preferably provided in advance of any immunodeficiency virus infection or symptom thereof. The prophylactic administration of the compound serves to prevent or attenuate any subsequent infection.

The compounds of the present invention can be administered in conventional solid or liquid pharmaceutical administration forms, for example, as uncoated or (film-) coated tablets, capsules, powders, granules, suppositories or solutions. The active substances can, for this purpose, be processed with conventional pharmaceutical aids such as tablet binders, fillers, preservatives, tablet disintegrants, flow regulators, plasticizers, wetting agents, dispersants, emulsifiers, solvents, sustained release compositions, antioxidants and/or propellant gases. The therapeutic compositions obtained in this way typically contain from about 0.1% to about 90% by weight of the active substance.

Having now generally described the invention, the same will be more readily understood through reference to the following examples, which are provided by way of illustration, and are not intended to be limiting of the present invention, unless specified.

### EXAMPLE 1

### Sigma-2 Receptor Agonists Inhibit HIV Infection of Lymphocytes by Reducing Membrane Sphingomyelin Content

HIV infection of CD4⁺ lymphocytes and release of virions occurs in lipid rafts; cholesterol- and sphingolipid-rich microdomains of the plasma membrane. Reducing membrane cholesterol content disrupts lipid rafts, and therefore has been found to also reduce HIV infectivity of lymphocytes and to diminish virulence of the virions released (Nguyen, D. H. et al. (2000) “EVIDENCE FOR BUDDING OF HUMAN IMMUNODEFIENCY VIRUS TYPE-1 SELECTIVELY FROM GLYCOLIPID-ENRICHED MEMBRANE LIPID RAFTS,” J. Virol. 74:3264-3272; Liao, Z. et al. (2001) “LIPID RAFTS AND HIV PATHOGENESIS: HOST MEMBRANE CHOLESTEROL IS REQUIRED FOR INFECTION BY HIV TYPE I,” AIDS Res. Hum. Retrovir. 17:1009-1019). Reducing membrane sphingomyelin also disrupts lipid rafts, and therefore would produce similar effects. Sigma-2 receptors are enigmatic receptors expressed in various tissues that recognize diverse psychoactive compounds. It has been found that sigma-2 receptor-activation reduces levels of membrane sphingomyelin in breast tumor cells (Crawford, K. W. et al. (2002) “SIGMA-2 RECEPTORS REGULATE CHANGES IN SPHINGOLIPID LEVELS IN BREAST TUMOR CELLS,” Eur. J. Pharmacol 443:207-209). Additionally, sigma-2 receptors were found to be localized to lipid rafts (Gebreselassie, D. et al. (2002) “Sigma-2 receptors are localized to sphingolipid/cholesterol-rich membrane rafts,” Proc. Amer. Assoc. Cancer Res. 43:725, #3597). In addition, sigma-2 receptor activation may inhibit P-1-3′ kinase signaling, an effect that should inhibit HIV infection of lymphocytes and macrophages (Francois, F. et al. (2002) “A ROLE FOR P13-KINASE SIGNALING DURING HIV INFECTION OF PRIMARY CD4+ T LYMPHOCYTES AND MACROPHAGES,” 9th Conference on Retroviruses and Opportunistic Infections, #171, 2002; http://www.retroconference.org/2002/Abstract/12694.htm).

Methods: Membrane sphingomyelin is metabolically labeled in H9 lymphocytes by incubating the cells with [³H] palmitic acid in supplemented RPMI media for 48 h. Labeled media is removed and cells are incubated in the presence or absence of various concentrations of the sigma-2 receptor agonists CB-184 or haldol (haloperidol) for 24 hours. Total lipids are extracted and the dried organic layer is chromatographed on Silica G-60 plates. Sphingomyelin spots are scraped and quantified by liquid scintillation counting.

CEMX174 cells transfected with a luciferase gene driven by the Simian Immunodeficiency Virus (SIV) LTR are used in infectivity assays. Cells are then treated with sigma-2 receptor agonists at various concentrations followed by incubation in the presence or absence (control) of HIV. Luciferase activity quantifies HIV replication.

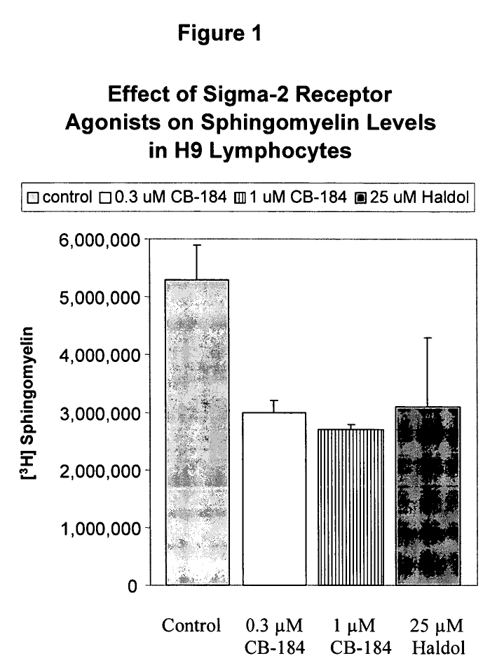

Results: Treatment of H9 cells with the sigma-2 receptor agonist CB-184 produced reductions of about 39% and 45% of control levels of membrane sphingomyelin at doses of 0.3 μM and 1 μM, respectively (FIG. 1). Similar results are obtained with haldol (FIG. 1). Haldol (haloperidol: Ortho McNeil Pharmaceutical Inc.) is a butyrophenone used in the treatment of Schizophrenia and other psychiatric conditions (Warraich P. S. et al. (2002) “HALOPERIDOL FOR THE ACUTE PHASE OF SCHIZOPHRENIA” Cochrane database system Rev. (3):CD001951; Lonergan, E. et al. (2002) “HALOPERIDOL FOR AGITATION IN DEMENTIA,” Cochrane Database Syst. Rev (2)CD002852). Haloperidol has been shown to be a sigma-2 agonist (Vilner B. J. et al. (2000) “MODULATION OF CELLULAR CALCIUM BY SIGMA-2 RECEPTORS: RELEASE FROM INTRACELLULAR STORES IN HUMAN SK-N-SH NEUROBLASTOMA CELLS,” J. Pharmacol. Exp. Ther. 292 (3): 900-11).

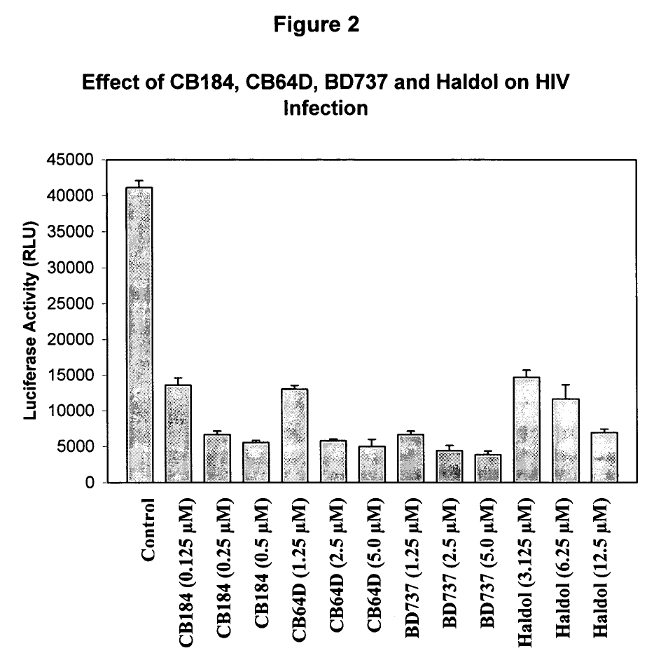

Structurally-diverse sigma-2 receptor agonists (CB-184, CB-64D, BD737, and haldol) produce dose-dependent inhibition of HIV infection (FIG. 2). This inhibition of HIV infection occurs at doses within the range of concentrations of CB-184 and haldol that produce reductions in membrane sphingomyelin, as shown above in FIG. 1. In these experiments, lymphocytes transfected with a luciferease gene driven by the Simian Immunodeficiency virus (SIV)LTR were incubated for 24 hours in the presence of the indicated dose of sigma-2 receptor agonist. The drugs were then removed, cells were washed and exposed to HIV for 24 hours. Cells were then lysed and luciferase activity quantified. Luciferase activity indicates the level of infectivity of the HIV virions. All of the compounds evaluated with sigma-2 agonist activity inhibited HIV infection at concentrations that were non-toxic to lymphocytes.

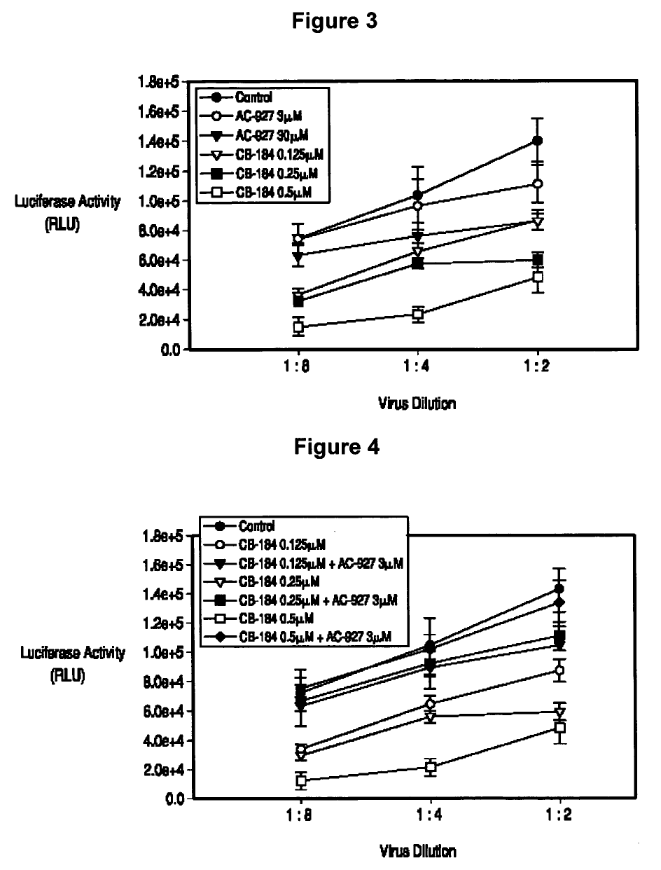

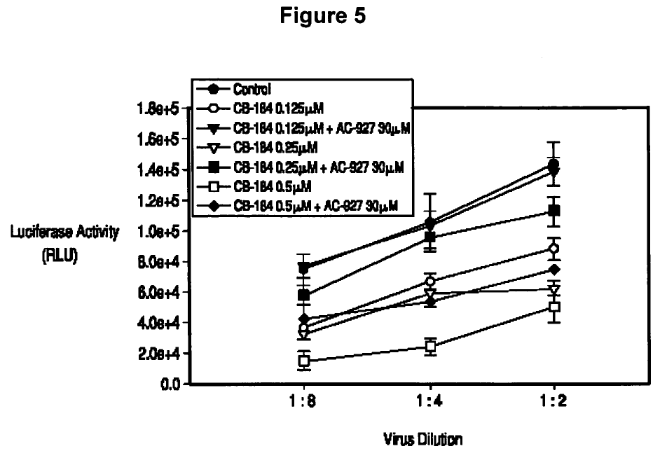

The sigma-2 receptor antagonist N-phenethylpiperidine (AC-927) reversed the ability of the potent sigma-2 agonist, CB-184, to inhibit HIV infection of lymphocytes. Different doses of the antagonist AC-927 abrogate the inhibitory actions of CB-184 at various doses and also with exposure to different HIV viral titres (FIG. 3, FIG. 4, FIG. 5). The ability of the antagonist to inhibit the effects of the sigma-2 receptor agonist provides strong evidence that the inhibition of infection is indeed mediated via the sigma-2 receptor.

### CONCLUSIONS

The results presented here indicate that structurally-diverse sigma-2 receptor agonists, at non-toxic concentrations, prevent HIV infection of lymphocytes. Such inhibition may be produced through the reduction of lymphocyte membrane sphingomyelin. This inhibitory action of sigma-2 receptor agonists can be reversed by selective receptor antagonists. Collectively, these findings support a role of sigma-2 receptors in HIV infection and support the use of sigma-2 agonists therapeutically in the treatment of HIV infection. This host-targeted intervention may circumvent the problems related to viral drug resistance providing which can have exceptional utility in treating HIV infection.

All publications and patents mentioned in this specification are herein incorporated by reference to the same extent as if each individual publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method for treating actual or potential infection by an immunodeficiency virus in a mammal, wherein said method comprises administering to said mammal a therapeutically or prophylactically effective amount of a sigma-2 agonist.

2. The method of claim 1, wherein said method is a method for treating actual infection by an immunodeficiency virus, and said administered amount of said sigma-2 agonist is a therapeutically effective amount.

3. The method of claim 1, wherein said method is a method for treating potential infection by an immunodeficiency virus, and said administered amount of said sigma-2 agonist is a prophylactically effective amount.

4. The method of claim 2, wherein said mammal is a human and said immunodeficiency virus is HIV.

5. The method of claim 3, wherein said mammal is a human and said immunodeficiency virus is HIV.

6. The method of claim 2, wherein said mammal is a feline and said immunodeficiency virus is a feline immunodeficiency virus.

7. The method of claim 3, wherein said mammal is a feline and said immunodeficiency virus is a feline immunodeficiency virus.

8. The method of claim 2, wherein said mammal is a simian and said immunodeficiency virus is a simian immunodeficiency virus.

9. The method of claim 3, wherein said mammal is a simian and said immunodeficiency virus is a simian immunodeficiency virus.

10. The method of claim 1, wherein said sigma-2 agonist is a sigma-2 selective agonist.

11. The method of claim 1, wherein said sigma-2 agonist is a (+)-5,8-disubstituted morphan-7-one, or a salt thereof.

12. The method of claim 1 wherein said (+)-5,8-disubstituted morphan-7-one is CB-184 {(+)-1R,5R^{-E-}8-(3,4-dichlorobenzylidene)-5-(3-hydroxyphenyl)-2-methylmorphan-7-one}.

13. The method of claim 2, wherein said method additionally comprises providing said mammal with a therapeutically effective amount of a drug that disrupts the life cycle of said virus, a drug that impairs the binding of said virus to a target cell, or a nucleotide analog.

14. The method of claim 3, wherein said method additionally comprises providing said mammal with a prophylactically effective amount of a drug that disrupts the life cycle of said virus, a drug that impairs the binding of said virus to a target cell, or a nucleotide analog.

15. An anti-immunodeficiency virus composition, said composition comprising an amount of a sigma-2 agonist effective for treating an actual or potential immunodeficiency virus infection in a mammal.

16. The anti-immunodeficiency virus composition of claim 15, wherein said sigma-2 agonist is a sigma-2 selective agonist.

17. The anti-immunodeficiency virus composition of claim 15, wherein said sigma-2 agonist is a (+)-5,8-disubstituted morphan-7-one, or a salt thereof.

18. The anti-immunodeficiency virus composition of claim 17, wherein said (+)-5,8-disubstituted morphan-7-one is CB-184 {(+)-1R,5R^{-E-}8-(3,4-dichlorobenzylidene)-5-(3-hydroxyphenyl)-2-methylmorphan-7-one}.

19. The anti-immunodeficiency virus composition of claim 15, wherein said composition additionally comprises a drug that disrupts the life cycle of said virus, a drug that impairs the binding of said virus to a target cell, or a nucleotide analog.

20. The anti-immunodeficiency virus composition of claim 15, wherein said virus is HIV, and said mammal is a human.
